(19) United States
(12) Patent Application Publication
(10) Pub. No.: US 2003/0119716 A1
(43) Pub. Date: Jun. 26, 2003

(54) Methods for screening, treating and diagnosing G-protein coupled receptor-related disorders and compositions thereof

(75) Inventors: Guyu Ho, Woburn, MA (US); Kathleen H. Young, Newtown, PA (US)

Correspondence Address: Bill T. Brazil, Five Giralda Farms, Madison, NJ, 07940-0874, US

(73) Assignee: Wyeth, Madison, NJ (US)

(21) Appl. No.: 10/215,776

(22) Filed: Aug. 9, 2002

Related U.S. Application Data
(60) Provisional application No. 60/311,684, filed on Aug. 10, 2001.

(51) Int. Cl. A61K 31/00; G01N 33/567; C12Q 1/68; A61K 31/70

(52) U.S. Cl. 514/1; 435/6; 435/7.21

(57) ABSTRACT

The present invention relates to novel methods and compositions for the diagnosis, treatment and prognosis of G-protein coupled receptor (GPCR)-related disorders through inhibition of regulators of G-protein signaling (RGS) proteins. The present invention relates to methods of screening and assessing test compounds useful in the intervention and prevention of GPCR-related disorders including neuropsychiatric and cardiopulmonary disorders. The invention further relates to methods to identify inhibitors for RGS expression or activity which are useful in the modulation of GPCR signaling pathways.

This application claims priority from copending provisional application serial No. 60/311,684, filed on Aug. 10, 2001, the entire disclosure of which is hereby incorporated by reference.

### FIELD OF THE INVENTION

The present invention is directed to novel methods for diagnosis, treatment and prognosis of G-protein coupled receptor (GPCR)-related disorders through inhibition of regulators of G-protein signaling (RGS) proteins. The present invention is further directed to methods of screening and assessing the efficacy of test compounds for the intervention and prevention of GPCR-related disorders and compositions capable of inhibiting GPCR-related disorders.

### BACKGROUND OF THE INVENTION

Many hormones, neurotransmitters, and sensory stimuli elicit specific physiological responses in target tissues by activating the cell surface receptors that are coupled to heterotrimeric G proteins, (See, e.g., Bourne et al., Nature (1990) 348:125-132; Hepler et al., Trends Biochem. Sci. (1992) 17: 383-387). Activated receptors promote exchange of GTP for GDP on Gα subunits leading to dissociation of active GTP-bound Gα from Gβγ dimers, both of which are signal transducers that activate an array of downstream signaling events. Signals are terminated following hydrolysis of GTP by Gα and the subsequent re-association of the Gβγ complex with the inactive GDP-bound Gα. Thus, the duration of the G-protein signaling depends on the rate of GTP hydrolysis and the rate of re-association of Gβγ.

The intrinsic GTP hydrolysis rate of Gα is too slow (about 1-5 minutes⁻¹) to explain the much faster deactivation rates of some G protein-controlled processes, such as phototransduction (Arshavsky et al., Neuron (1998) 20:11-14) and ion channel activation (See, Kurachi, Am. J. Physiol. (1995) 269:C821-C830). The discrepancy is accounted for by the recent discovery of a large family of RGS proteins (See, Zerangue et al., Cur. Biol. (1998) 8:313-316; Berman et al., J. Biol. Chem. (1998) 273:1269-1272; Hepler, Trends Biochem. Sci. (1999) 17:383-387). RGS proteins act in part as Gα GAPs that shorten the half-life of the active GTP-bound Gα, thus attenuating responses generated from both Gα-GTP and free Gβγ (Zhong and Neubig J. Pharma. Exp. Thera. (2001) 297:837-845). The GAP activity of RGS proteins is conferred by the conserved RGS core domain of about 120 amino acids. The crystal structure of an RGS and Gα complex illustrates that the RGS core binds to the flexible switch regions of Gα, thereby facilitating the GTP hydrolysis by stabilizing the transition state (Tesmer et al., Cell (1997) 89:251-261).

In vitro biochemical studies show that RGS proteins exhibit differential GAP activities for the Gαq and Gαi classes of proteins (De Vries and Farquar, Trends Cell Biol. (1999) 9:138-143). For example, RGS2 only binds Gαq and inhibits Gαq-directed activation of phospholipase C (Heximer et al., Proc. Natl. Acad. Sci. (1997) 94:14389-14393). RGS4, on the other hand, binds both Gαi and Gαq and accelerates the hydrolysis of Gαi and additionally inhibits Gαq-directed activation of phospholipase C (Hepler et al., (1997) supra). While both RGS2 and RGS4 are Gαq GAPs, they differ quantitatively in their activity, with RGS2 more potent in blocking Gαq-directed activation of phospholipase C. RGSz1 binds Gαz, a member of Gαi family, and is at least 100-fold more selective for Gαz than other members of Gαi family in accelerating GTP hydrolysis (Wang et al., J. Biol. Chem. (1997) 273:26014-26025; Glick et al., J. Biol. Chem. (1997) 273:26008-26013) While there is a correlation between the in vitro Gα selectivity of RGS proteins and the in vivo selective attenuation of G protein signaling in some cell systems (Huang et al., Proc. Natl. Acad. Sci. (1997) 94:6159-6163; Dunpnik, et al., Proc. Natl. Acad. Sci. (1997) 94:10461-10466; Bowman et al., J. Biol. Chem. (1998) 273:28040-28048; Heximer et al., J. Biol. Chem. (1999) 274:34253-34259), the discrepancy is apparent in others. For example, RGS2 inhibits both Gαq and Gαi-coupled MAPK activation in transfected COS cells (Ingi et al., J. Neurosci. (1998) 18:7178-7188). Moreover, RGS2 inhibits Gi-coupled melanophore pigment dispersion more potently than RGS4 (Potenza et al., J. Pharm. Exp. Thera. (1999) 291:482-491).

SRE (Serum Response Element) is a regulatory sequence found in many growth factor-regulated promoters (Treisman, Semin. Cancer Biol. (1990) 1:47-58). SRE binds the ubiquitous transcription factor SRF (serum response factor) that is required for the SRE activity (Norman et al., Cell (1988) 55:989-1003). At the c-fos SRE, SRF forms a ternary complex with TCF (ternary complex factor), which is comprised of members of a small family of transcription factors, including Elk1 (Shaw et al., Cell (1989) 56:563-572). The TCF binds a recognition motif adjoining the SRF-binding site and regulates SRE activity in response to activation of the Ras-Raf-Erk pathway (Treisman, Curr. Opin. Genet. Dev. (1990) 4:96-101; Kortenjann et al., Mol. Cell Biol. (1994) 14:4815-4824). The c-fos SRE activation is induced cooperatively or independently by the SRF-linked and TCF-linked pathways (Hill et al., Cell (1995) 81:1159-1170). Expression of constitutively active Gαq or Gα_{12/13} induces activation of an SRE-reporter gene in cultured cells and the activation is mediated via the SRF-linked pathway (Fromm et al., Proc. Natl. Acad. Sci. (1997) 94:10098-10103; Mao et al., J. Biol. Chem. (1998) 273:27118-27123). Expression of Gβγ dimers in cells also activates the SRE-reporter gene and Gβγ-induced activation is believed to be mediated through the TCF-linked pathway.

Accordingly, regulators of G protein signaling (RGS) proteins function as GTPase-activating proteins (GAPs) to inhibit the G protein coupled receptor signaling initiated by both Gα-GTP and Gβγ. While certain RGS proteins are selective for Gα GAPs in vitro, their in vivo selectivity is unclear. Accordingly, there is a need in the art for novel methods and compositions which provide diagnostics, prognostics and therapeutics based on in vivo signaling. The present invention provides such methods and compositions. The present invention also provides novel drug screening and drug efficacy methods.

### SUMMARY OF THE INVENTION

In one embodiment, the invention provides a method of assessing the efficacy of a test compound for inhibiting a GPCR-related disorder in a subject by contacting a test cell with one of a plurality of test compounds in the presence of a GPCR agonist, where the test cell comprises a GPCR, a RGS protein, a corresponding Gα protein that is expressed at a level capable of attenuating GPCR signaling by at least 50% as compared to a cell without the Gα protein expression level and a reporter gene. The method continues by detecting the expression of the reporter gene in the test cell contacted by a test compound and comparing the expression of the reporter gene in the test cell contacted by the test compound with the expression of the reporter gene in a test cell contacted by the agonist in the absence of the test compound, wherein a substantially increased level of expression of the reporter gene in the test cell contacted by the test compound and agonist, relative to the expression of the reporter gene in the test cell contacted by the agonist in the absence of the test compound, is an indication that the test compound is efficacious for inhibiting the GCPR-related disorder in the subject.

In a preferred embodiment, the GPCR-related disorder is a neuropsychiatric disorder or a cardiovascular disorder. In another preferred embodiment, the GPCR is a D2 receptor, M2 receptor, 5HTIA receptor, Edg1 receptor or Bradykinin receptor. In another preferred embodiment, the RGS protein is GAIP, RGSz1, RGS1, RGS2, RGS3, RGS4, RGS5, RGS6, RGS7, RGS8, RGS9, RGS10, RGS11, RGS13, RGS14, RGS16, RGS17, D-AKAP2, p115RhoGEF, PDZ-RhoGEF, bRET-RGS, Axin, and mCONDUCTIN. In a further preferred embodiment, the reporter gene is SRE-Luciferase, SRE-LacZ, SRE-CAT or CRE-Luciferase. In still another preferred embodiment, the Gα protein is Gαi or Gαq. More preferably, the Gαi protein is either Gαi1, Gαi2, Gαi3, Gαz or Gαo. In still another preferred embodiment, the Gα protein is a chimeric protein. More preferably, the chimeric protein is a chimeric protein between Gαq and Gαi1. In another preferred embodiment, the test cell expresses wild type signaling molecules of the Ras-Raf-MEK pathway. More preferably, the signaling molecules of the Ras-Raf-MEK pathway are Ras, Raf, MEK, Erk_{1/2}, Elk₁, JNK and p38. In another preferred embodiment, the test cell expresses wild type Rho family molecules. More preferably, the Rho family members are RhoA, Rac1, and Cdc42. in another preferred embodiment, the Gα protein is transiently transfected into the test cells. In still another preferred embodiment, the reporter gene is transiently transfected into the test cells. In still another preferred embodiment, the GPCR is stably transfected into the test cells.

In another embodiment, the invention provides a method of assessing the efficacy of a test compound for inhibiting a GPCR-related disorder in a subject by comparing expression of a RGS protein in the presence of Gα in a first cell sample, where the first cell sample is exposed to the test compound, and expression of a RGS protein in the presence of Gα in a second cell sample, where the second cell sample is not exposed to the test compound, where a substantially decreased level of expression of the RGS protein in the first sample, relative to the second sample, is an indication that the test compound is efficacious for inhibiting the GPCR-related disorder in the subject. Preferably, the GPCR-related disorder is a neuropsychiatric disorder or cardiovascular disorder. In another preferred embodiment, the RGS protein is GAIP, RGSz1, RGS1, RGS2, RGS3, RGS4, RGS5, RGS6, RGS7, RGS8, RGS9, RGS10, RGS11, RGS13, RGS14, RGS16, RGS17, D-AKAP2, p115RhoGEF, PDZ-RhoGEF, bRET-RGS, Axin, or mCONDUCTIN. In another preferred embodiment, the Gα protein is Gαi or Gαq. More preferably, the Gαi protein is Gαi1, Gαi2, Gαi3, Gαz or Gαo.

In another embodiment, the present invention provides a method of high-throughput screening for test compounds capable of inhibiting an RGS protein by contacting a test cell with one of a plurality of test compounds in the presence of a GPCR agonist, where the test cell includes a GPCR, an RGS protein, a corresponding Gα protein expressed at a level capable of attenuating GPCR-signaling by at least 50% as compared to a cell without said Gα protein expression level, and a reporter gene. The method also includes the steps of detecting the expression of the reporter gene in the test cell contacted by a test compound relative to other test compounds, and correlating the amount of expression level of the reporter gene with the ability of the test compound to inhibit RGS protein, where increased expression of the reporter gene indicates that the test compound is capable of inhibiting the RGS protein. In a preferred embodiment, the GPCR is a D2 receptor, M2 receptor, 5HTIA receptor, Edg1 receptor or Bradykinin receptor. In another preferred embodiment, the RGS protein is GAIP, RGSz1, RGS1, RGS2, RGS3, RGS4, RGS5, RGS6, RGS7, RGS8, RGS9, RGS10, RGS11, RGS13, RGS14, RGS16, RGS17, D-AKAP2, p115RhoGEF, PDZ-RhoGEF, bRET-RGS, Axin, or mCONDUCTIN. In another preferred embodiment, the reporter gene is SRE-Luciferase, SRE-LacZ, SRE-CAT or CRE-Luciferase. In another preferred embodiment, the Gα protein is Gαi or Gαq. More preferably, the Gα protein is Gαi1, Gαi2, Gαi3, Gαz or Gαo. In another preferred embodiment, the Gα protein is a chimeric protein. In another preferred embodiment, the test cell includes wild type signaling molecules of the Ras-Raf-MEK pathway. More preferably, the signaling molecules of the Ras-Raf-MEK pathway include Ras, Raf, MEK, Erk_{1/2}, Elk₁, JNK and p38. In another preferred embodiment, the test cell includes the wild type Rho family molecules. More preferably, the Rho family molecules include RhoA, Rac1, and Cdc42. In another preferred embodiment, the test compounds are bioactive agents such as naturally-occurring compounds, biomolecules, proteins, peptides, oligopeptides, polysaccharides, nucleotides or polynucleotides. Alternatively, the test compounds are small molecules.

In another embodiment, the invention provides a method of high-throughput screening for test compounds capable of inhibiting a GPCR-related disorder in a subject by combining an RGS protein, Gα, and a test compound; detecting binding of the RGS protein and Gα in the presence of a test compound; and correlating the amount of inhibition of binding between RGS and Gα with the ability of the test compound to inhibit the GPCR-related disorder, where inhibition of binding of the RGS protein and Gα indicates that the test compound is capable of inhibiting the GPCR-related disorder. In a preferred embodiment, the test compounds are small molecules. Alternatively, the test compounds are bioactive agents, such as naturally-occurring compounds, biomolecules, proteins, peptides, oligopeptides, polysaccharides, nucleotides or polynucleotides. In another preferred embodiment, the Gα protein is Gαi or Gαq. More preferably, the Gαi protein is Gαi1, Gαi2, Gαi3, Gαz or Gαo.

In another embodiment, the invention provides a method of screening test compounds for inhibitors of a GPCR-related disorder in a subject by obtaining a sample from a subject comprising cells; contacting an aliquot of the sample with one of a plurality of test compounds; detecting the expression level of an RGS protein and Gα in each of the aliquots, and selecting one of the test compounds which substantially inhibits expression of a RGS protein in the aliquot containing that test compound, relative to other test compounds. In a preferred embodiment, the Gα is Gαi or Gαq. More preferably, the Gαi is Gαi1, Gαi2, Gαi3, Gαz or Gαo.

In another embodiment, the invention provides a method of screening test compounds for inhibitors of a GPCR-related disorder in a subject by obtaining a sample from a subject comprising cells; contacting an aliquot of the sample with one of a plurality of test compounds; detecting the activity of an RGS protein in each of the aliquots; and selecting one of the test compounds which substantially inhibits expression of a RGS protein in the aliquot containing that test compound, relative to other test compounds. In a preferred embodiment, the Gα is Gαi or Gαq. More preferably, the Gαi is Gαi1, Gαi2, Gαi3, Gαz or Gαo.

In another embodiment, the invention provides a method of screening for a test compounds capable of interfering with the binding of an RGS protein and a Gα by combining an RGS protein, a test compound, and a Gα; determining the binding of the RGS protein and the Gα; and correlating the ability of the test compound to interfere with binding, where a decrease in binding of the RGS protein and the Gα in the presence of the test compound as compared to the absence of the test compound indicates that the test compound is capable of inhibiting binding. In a preferred embodiment, the test compound is a small molecule. More preferably, the test compound are bioactive agents, such as naturally-occurring compounds, biomolecules, proteins, peptides, oligopeptides, polysaccharides, nucleotides or polynucleotides. Alternatively, the test compound is a protein. In another embodiment, the Gα protein is Gαi or Gαq. More preferably, the Gαi protein is Gαi1, Gαi2, Gαi3, Gαz or Gαo. Alternatively, the Gα protein is a chimeric protein.

In another embodiment, the present invention provides a method of determining the severity of a GPCR-related disorder in a subject by comparing a level of expression of an RGS protein in a sample from the subject; and a normal level of expression of an RGS protein in a control sample where an abnormal level of expression of the RGS protein in the sample from the subject relative to the normal levels is an indication that the subject is suffering from a severe GPCR-related disorder. In a preferred embodiment, the presence of the RGS protein is detected using an antibody, or fragments thereof, which specifically binds to the RGS protein. in another preferred embodiment, the control sample is collected from tissue substantially free of the GPCR-related disorder and the abnormal level of expression is by a factor of at least about 2.

In another embodiment, the present invention provides a method of assessing the efficacy of a therapy for inhibiting a GPCR-related disorder in a subject by comparing the expression of a RGS protein in a first sample obtained from the subject prior to providing at least a portion of the therapy to the subject, and expression of a RGS protein in a second sample following provision of the portion of the therapy where a substantially modulated level of expression of the RGS protein in the second sample, relative to the first sample, is an indication that the therapy is efficacious for inhibiting the GPCR-related disorder in the subject.

In another embodiment, the present invention provides a method for diagnosing a GPCR-related disorder by obtaining a sample from a subject comprising cells; measuring the expression of RGS and Gα in the sample, correlating the amount of RGS and Gα with the presence of a GPCR-related disorder, where the substantially increased levels of RGS and Gα as compared to a control sample are indicative of the presence of GPCR-related disorder.

In another embodiment, the present invention provides a method of treating a subject diagnosed with a GPCR-related disorder by administering a composition including an RGS inhibitor which specifically binds to an RGS protein; a Gα inhibitor which specifically binds to a Gα protein; and a pharmaceutically acceptable carrier. In a preferred embodiment, the RGS inhibitor and the Gα inhibitor are small molecules. In a more preferred embodiment, the RGS inhibitor and the Gα inhibitor are polypeptides. In another preferred embodiment, the RGS inhibitor and the Gα inhibitor are polynucleotides.

In another embodiment, the present invention provides a method of treating a subject diagnosed with a GPCR-related disorder by administering a composition including an antisense oligonucleotide complementary to an RGS polynucleotide; an antisense oligonucleotide complementary to a Gα polynucleotide; and a pharmaceutically acceptable carrier. In a preferred embodiment, the antisense oligonucleotide is complementary to an RGS polynucleotide such as, for example, GAIP, RGSz1, RGS1, RGS2, RGS3, RGS4, RGS5, RGS6, RGS7, RGS8, RGS9, RGS10, RGS11, RGS13, RGS14, RGS16, RGS17, D-KAP2, p115RhoGEF, PDZ-RhoGEF, bRET-RGS, Axin, or mCONDUCTIN. In another preferred embodiment, the Gα protein is Gαi or Gαq. More preferably, the Gαi protein is Gαi1, Gαi2, Gαi3, Gαz or Gαo.

In another embodiment, the present invention provides a method of treating a subject diagnosed with a GPCR-related disorder by administering a composition including a ribozyme which is capable of binding an RGS polynucleotide; a ribozyme which is capable of binding a Gα polynucleotide; and a pharmaceutically acceptable carrier. In a preferred embodiment, the RGS polynucleotide encodes a GAIP, RGSz1, RGS1, RGS2, RGS3, RGS4, RGS5, RGS6, RGS7, RGS8, RGS9, RGS10, RGS11, RGS13, RGS14, RGS16, RGS17, D-AKAP2, p115RhoGEF, PDZ-RhoGEF, bRET-RGS, Axin, mCONDUCTIN polynucleotide or polynucleotide sequence for RGS proteins disclosed in U.S. Pat. No. 6,069,296 or U.S. Pat. No. 5,929,207, the disclosures of which are herein incorporated by reference. In another preferred embodiment, the Gα polynucleotide is a Gαi and Gαq polynucleotide. More preferably, the Gαi polynucleotide is a Gαi1, Gαi2, Gαi3, Gαz or Gαo polynucleotide.

In another embodiment, the present invention provides a method of enhancing GPCR-signaling by providing to cells of a subject an antisense oligonucleotide complementary to an RGS polynucleotide. In a preferred embodiment, the antisense oligonucleotide is complementary to a GAIP, RGSz1, RGS1, RGS2, RGS3, RGS4, RGS5, RGS6, RGS7, RGS8, RGS9, RGS10, RGS11, RGS13, RGS14, RGS16, RGS17, D-AKAP2, p115RhoGEF, PDZ-RhoGEF, bRET-RGS, Axin, or mCONDUCTIN polynucleotide.

In another embodiment, the present invention provides a method of inhibiting GPCR-signaling, the method comprising providing to cells of a subject an antisense oligonucleotide complementary to Gα. In a preferred embodiment, the Gα protein is Gαi or Gαq. Preferably, the Gαi protein is Gαi1, Gαi2, Gαi3, Gαz or Gαo.

In another embodiment, the invention provides a composition capable of inhibiting a GPCR-related disorder in a subject, where the composition includes a therapeutically effective amount of an RGS inhibitor which specifically binds to an RGS protein; a Gα inhibitor which specifically binds to a Gα protein; and a pharmaceutically acceptable carrier.

In another embodiment, the invention provides a composition capable of inhibiting a GPCR-related disorder where the composition includes a therapeutically effective amount of an antisense oligonucleotide complementary to an RGS polynucieotide and an antisense oligonucieotide complementary to a Gα polynucleotide; and a pharmaceutically acceptable carrier.

In another embodiment, the invention provides a composition capable of inhibiting a GPCR-related disorder where the composition includes a therapeutically effective amount of a ribozyme which is capable of binding an RGS polynucleotide; a ribozyme which is capable of binding a Gα polynucleotide; and a pharmaceutically acceptable carrier.

In another embodiment, the invention provides a genetically engineered test cell including a GPCR, a RGS protein, a corresponding Gα protein expressed at a level capable of attenuating GPCR-signaling by at least 50% as compared to a cell without said Gα protein expression level, and a reporter gene, where at least one of the components is introduced into the cell. In a preferred embodiment, the test cell is a mammalian cell. In another preferred embodiment, the GPCR is a dopamine receptor (D2 or D2R). In another preferred embodiment, the RGS protein is an RGS2, RGS4 or RGSz protein. In another preferred embodiment, the corresponding Gα protein is a Gαi protein. In another preferred embodiment, the corresponding Gα protein is a Gαq/i chimeric protein.

In another embodiment, the invention provides a kit for determining the long term prognosis in a subject having a GPCR-related disorder where the kit includes a first polynucleotide probe, where the probe specifically binds to a transcribed RGS polynucleotide, and a second polynucleotide probe, where the probe specifically binds to a transcribed Gα polynucleotide.

In another embodiment, the invention provides a kit for determining the long term prognosis in a subject having a GPCR-related disorder where the kit includes a first antibody, where the first antibody specifically binds to a RGS polypeptide, and a second antibody, where the second antibody specifically binds to a corresponding Gα polypeptide.

In another embodiment, the invention provides a kit for assessing the suitability of each of a plurality of compounds for inhibiting a GPCR-related disorder in a subject where the kit includes a plurality of test cells, where each test cell includes a GPCR, a RGS protein, a corresponding Gα protein expressed at a level capable of attenuating GPCR-signaling by at least 50% as compared to a cell without said Gα protein expression level, and a reporter gene. The kit also includes an agonist for the GPCR.

### BRIEF DESCRIPTION OF THE DRAWINGS

FIG. 1 demonstrates that quinpirole (QUIN) stimulates c-fos SRE activation. Quinpirole stimulates the c-fos SRE activation and the activity is abrogated by pertussis-toxin (PTX) and βARKct. CHO-D2R cells were transiently transfected with pSRE-Luc (1 μg) and pβGal (10 ng) reporter constructs in the presence of βARKct or control plasmid (4 μg). Thereafter, cells were serum-starved overnight in the presence or absence of 10 ng/ml of PTX prior to treatment with 10 μM quinpirole for 5 hours. The luciferase activity (reflecting SRE activation) was measured and normalized with the β-Gal activity. The numbers shown are representative of at least two independent experiments conducted in triplicate.

FIG. 2 shows the effect of RGS proteins on quinpirole-stimulated SRE activation. CHO-D2R cells were transiently transfected with pSRE-Luc (2 μg), pβGal (10 ng), the indicated RGS proteins or vector (2 μg), and additional vector plasmid to total of 5 μg DNA used in each transfection. After serum-starvation overnight, cells were treated with 0 nM, 10 nM, 100 nM, 1 μM, 10 μM, and 100 μM of quinpirole for 5 hours before measuring luciferase and β-Gal activities. The numbers shown represent at least two independent experiments, each conducted in triplicate. Standard errors were within 5% of the corresponding values.

FIG. 3A and 3B show the expression of Gα proteins potentiated inhibition of RGS proteins on quinpirole-stimulated SRE activation.

FIG. 3A: Comparison of RGS4 Activity in the Presence or Absence of Gαi1Co-Transfection. CHO-D2R cells were transiently transfected with pSRE-Luc (2 μg), pβGal (10 ng), RGS4 (2 μg), and Gαi1 or vector (1 μg). Cells were then serum-starved overnight, treated with 100 nM quinpirole for 5 hours, after which, luciferase and β-Gal activity was measured.

FIG. 3B: Differential Potentiation by Gαi1 on the Activity of RGS Proteins. CHO-D2R cells were transiently transfected with pSRE-Luc (2 μg), pβbGal (10 ng), Gαi1 (1 μg), and the indicated RGS proteins or vector (2 μg). Cells were then serum-starved overnight, treated with 0 nM, 10 nM, 100 nM,1 μM, 10 μM, and 100 μM of quinpirole for 5 hours prior to measuring luciferase and β-Gal activities

FIG. 3C: Gαq/i Chimera Potentiated the Activity of Both RGS2 and RGS4. The experiment was performed in an identical manner as in FIG. 3B except that Gαq/i chimera was used in place of Gαi1 and quinpirole concentrations were one order of magnitude lower. The numbers shown represent at least two independent experiments, each with triplicate transfections. Standard errors were within 2% of the corresponding values.

FIG. 4 shows PD098059 inhibited quinpirole-stimulated Erk1/2 activation and SRE activation. CHO-D2R cells were transiently transfected with pSRE-Luc (1 μg) and pβGal (10 ng) reporter genes and control plasmids to make up 5 μg of total DNA used per each transfection. After serum-starvation overnight, cells were treated with 25 nM PD098059 or vehicle for 30 minutes before addition of 100 nM quinpirole. After a 5-min incubation with quinpirole, cells were lysed and the lysates analyzed by Western blot with anti-phospho-Erk1/2 antibodies. The blot was stripped and re-probed with anti-Erk1/2 antibodies to show the total protein loading. Luciferase and β-Gal activities were measured after incubation with quinpirole for 5 hours. Numbers shown represent at least two independent experiments, each with triplicate transfections.

FIG. 5 demonstrates that dominant negative mutants of RhoA, Rac1, and Cdc42 inhibit quinpirole-stimulated SRE activation. CHO-D2R cells were transiently transfected with pSRE-Luc (2 μg), pβGal (10 ng), RhoN19 or RacN17 or Cdc42N17 or vector (3 μg). After serum-starvation overnight, cells were treated with 100 nM quinpirole for 5 hours before measuring luciferase and β-Gal activities. The numbers shown represent at least two independent experiments, each with triplicate transfections.

FIG. 6 shows that Wortmannin had no effect on quinpirole-stimulated SRE activation. The experiments were performed in an identical manner as described in FIG. 4 except that 50 nM wortmannin was used in place of PD098059 and the Western blot was probed with either anti-phospho-Akt or anti-phospho-Erk1/2 antibodies, stripped, and re-probed with anti-Akt or anti-Erk1/2 antibodies to show the total protein loading.

### DETAILED DESCRIPTION OF THE INVENTION

The present invention provides novel methods for screening, treating and diagnosing GPCR-related disorders. The present invention also provides novel compositions for treating and inhibiting GPCR-related disorders.

Definitions and Terms

To facilitate an understanding of the present invention, a number of terms and phrases are defined below.

As used herein, the term “GPCR-signaling molecule” includes a polynucleotide or polypeptide molecule which is increased or decreased in quantity or activity in GPCR-containing cells treated with a GPCR agonist as compared to GPCR-containing cells not treated with an agonist or which is known in the art to transduce a signal either directly or indirectly from a GPCR to one or more cellular proteins or molecules. In certain embodiments, the GPCR-signaling molecules of the invention include, but are not limited to, Ras, Raf, MEK, Erk_{1/2}, JNK, p38 and Elk₁, as well as homologs or isoforms thereof, particularly human homologs or human isoforms. In certain embodiments, GPCR-signaling molecules comprise a GPCR-signaling pathway.

As used herein, the term “RGS” or “RGS protein” includes regulators of G protein signaling now known, or later described, which are capable of inhibiting or binding to a Gαi class protein or a Gαq class protein. Such RGS proteins include, but are not limited to, GAIP, RGSz1, RGS1, RGS2, RGS3, RGS4, RGS5, RGS6, RGS7, RGS8, RGS9, RGS10, RGS11, RGS13, RGS14, RGS16, RGS17, D-AKAP2, p115RhoGEF, PDZ-RhoGEF, bRET-RGS, Axin, and mCONDUCTIN, as well as any now known, or later described, isoforms or homologs. For example, several isoforms of RGS9 are known and described in Cowan et al., (2001) Prog. Nuc. Acids Res. 65:341-359, incorporated herein by reference. Additionally, as used herein, the term “RGS” includes now known, or later described, protein that contain an RGS core domain (see, e.g., Dohlman et al., (1997) J. Biol. Chem. 272:3871-3874; Berman et al., (1998) J. Biol. Chem. 273:1269-1272; Zheng et al., (1999) Trends Biol. Sci. 24:411-414; DeVries et al., (2000) Ann. Rev. Pharm. Toxicol. 40:235-271). Generally RGS proteins contain an RGS core domain (such as described in Berman et al., (1998) J. Biol. Chem. 273:1269-72), however, in certain embodiments, an RGS polypeptide or polynucleotide encoding an RGS polypeptide may contain one or more mutations, deletions or insertions In such embodiment, the RGS protein core domain is at least 60% homologous, preferably 75% homologous, more preferably 85% or more homologous, to a wild type core domain.

As used herein, the term “Gα” or “Gα protein” of the invention includes all members of the Gαi class or Gαq class now known or later described, including but not limited to Gαi1, Gαi2, Gαi3, Gαz, Gαo and Gαq. In certain embodiments, a Gα protein of the invention may contain one or more mutations. deletions or insertions. In such embodiments, the Gα protein is at least 60% homologous, preferably 75% homologous, more preferably 85% or more homologous, to a wild type Gα protein.

As used herein, the term “corresponding Gα protein” means a Gα protein which is capable of contacting an RGS protein in the cell, screening assay or system in use. Corresponding Gα proteins are also coupled to the GPCR in the cell, screening assay or system in use such that the Gα protein is capable of contacting the GPCR or is capable of transducing a signal in response to agonist binding to the GPCR. In certain embodiments the corresponding Gα protein is capable of contacting a specific RGS as set forth in the non-limiting examples shown in Table 1.

TABLE 1

| | | |
|---|---|---|
| GAIP | RGSz1 | RGS1 |
| RGS2 | RGS3 | RGS4 |
| RGS5 | RGS6 | RGS7 |
| RGS8 | RGS9 | GS10 |
| RGS11 | RGS13 | RGS14 |
| RGS16 | RGS17 | D-AKAP2 |
| p115RhoGEF | PDZ-RhoGEF | bRET-RGS |
| Axin | mCONDUCTIN | |

In a specific embodiment of the invention, the corresponding Gα protein is a Gαq protein which is capable of contacting an RGS2 protein. In another specific embodiment of the invention, the corresponding Gα protein is a Gαi protein which is capable of contacting an RGS4 protein. In another specific embodiment of the invention, the corresponding Gα protein is a Gαq protein which is capable of contacting an RGS4 protein. In yet another specific embodiment of the invention, the corresponding Gα protein is a Gαz protein which is capable of contacting an RGSz protein.

As used herein, the term “GPCR-related disorder” includes any disease or disorder associated with aberrant GPCR signaling, including, but not limited to, neuropsychiatric disorders such as, for example, schizophrenia, bipolar disorders and depression; cardiopulmonary disorders such as, for example, cardiachypertrophy, hypertension, thrombosis and arrhythmia; inflammation, cystic fibrosis and ocular disorders. Without limitation as to mechanism, GPCR-related disorders are generally associated with decreased GPCR-signaling.

As used herein, the term “GPCR agonist” includes any molecule or agent which binds to a GPCR and elicits a response. As used herein, the term “GPCR antagonist” includes any molecule or agent which binds to a GPCR but which does not elicit a response.

As used herein, the terms “polynucleotide,” “nucleic acid” and “oligonucleotide” are used interchangeably, and include polymeric forms of nucleotides of any length, either deoxyribonucleotides or ribonucleotides, or analogs thereof. Polynucleotides may have any three-dimensional structure, and may perform any function, known or unknown. The following are non-limiting examples of polynucleotides: a gene or gene fragment, exons, introns, messenger RNA (mRNA), transfer RNA, ribosomal RNA, ribozymes, DNA, cDNA, genomic DNA, recombinant polynucleotides, branched polynucleotides, plasmids, vectors, isolated DNA of any sequence, isolated RNA of any sequence, nucleic acid probes, and primers. Polynucleotides of the invention may be naturally-occurring, synthetic, recombinant or any combination thereof. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also includes both double- and single-stranded molecules. Unless otherwise specified or required, any embodiment of this invention that is a polynucleotide encompasses both the double-stranded form and each of two complementary single-stranded forms known or predicted to make up the double-stranded form.

The term “polynucleotide sequence” is the alphabetical representation of a polynucleotide molecule. A polynucleotide is composed of a specific sequence of four nucleotide bases: adenine (A); cytosine (C); guanine (G); thymine (T); and uracil (U) in place of guanine when the polynucleotide is RNA This alphabetical representation can be inputted into databases in a computer and used for bioinformatics applications such as, for example, functional genomics and homology searching.

The term “isolated polynucleotide molecule” includes polynucleotide molecules which are separated from other polynucleotide molecules which are present in the natural source of the polynucleotide. For example, with regard to genomic DNA, the term “isolated” includes polynucleotide molecules which are separated from the chromosome with which the genomic DNA is naturally associated. Preferably, an “isolated” polynucleotide is free of sequences which naturally flank the polynucleotide (i.e., sequences located at the 5′ and 3′ ends of the polynucleotide of interest) in the genomic DNA of the organism from which the polynucleotide is derived. For example, in various embodiments, the isolated polynucleotide molecule of the invention, or polynucleotide molecule encoding a polypeptide of the invention, can contain less than about 5 kb, 4 kb, 3 kb, 2 kb, 1 kb, 0.5 kb or 0.1 kb of nucleotide sequences which naturally flank the polynucleotide molecule in genomic DNA of the cell from which the polynucleotide is derived. Moreover, an “isolated” polynucleotide molecule, such as a cDNA molecule, can be substantially free of other cellular material, or culture medium when produced by recombinant techniques, or substantially free of chemical precursors or other chemicals when chemically synthesized.

A “gene” includes a polynucleotide containing at least one open reading frame that is capable of encoding a particular polypeptide or protein after being transcribed and translated. Any of the polynucleotide sequences described herein may also be used to identify larger fragments or full-length coding sequences of the gene with which they are associated. Methods of isolating larger fragment sequences are known to those of skill in the art.

As used herein, a “naturally-occurring” polynucleotide molecule includes, for example, an RNA or DNA molecule having a nucleotide sequence that occurs in nature (e.g., encodes a natural protein).

As used herein, the term “transcribed” or “transcription” refers to the process by which genetic code information is transferred from one kind of nucleic acid to another, and refers in particular to the process by which a base sequence of mRNA is synthesized on a template of cDNA.

The term “polypeptide” includes a compound of two or more subunit amino acids, amino acid analogs, or peptidomimetics. The subunits may be linked by peptide bonds. In another embodiment, the subunit may be linked by other bonds, e.g., ester, ether, etc. As used herein, the term “amino acid” includes either natural and/or unnatural or synthetic amino acids, including glycine and both the D or L optical isomers, and amino acid analogs and peptidomimetics. A peptide of three or more amino acids is commonly referred to as an oligopeptide. Peptide chains of greater than three or more amino acids are referred to as a polypeptide or a protein.

A “gene product” includes mRNA generated when a gene is transcribed or a polypeptide generated when a gene is transcribed and translated.

As used herein, a “chimeric protein” or “fusion protein” comprises a first polypeptide operatively linked to a second polypeptide. Chimeric proteins may optionally comprise a third, fourth or fifth or other polypeptide operatively linked to a first or second polypeptide. Chimeric proteins may comprise two or more different polypeptides. Chimeric proteins may comprise multiple copies of the same polypeptide. Chimeric proteins may also comprise one or more mutations in one or more of the polypeptides. Methods for making chimeric proteins are well known in the art. In one embodiment of the invention, the chimeric protein is a chimera of Gαi and Gαq.

An “isolated” or “purified” protein, polynucleotide or molecule means substantially free of cellular material, such as other contaminating proteins from the cell or tissue source from which the protein polynucleotide or molecule is derived, or substantially free from chemical precursors or other chemicals when chemically synthesized. The language “substantially free of cellular material” includes preparations separated from cellular components of the cells from which it is isolated or recombinantly produced or synthesized. In one embodiment, the language “substantially free of cellular material” includes preparations of a protein of interest having less than about 30% (by dry weight) of other proteins (also referred to herein as a “contaminating protein”), more preferably less than about 20%, still more preferably less than about 10%. and most preferably less than about 5% of other proteins. When the protein or polynucieotide is recombinantly produced, it is also preferably substantially free of culture medium, i.e., culture medium represents less than about 20%, more preferably less than about 10%, and most preferably less than about 5% of the volume of the preparation of the protein of interest.

The language “substantially free of chemical precursors or other chemicals” includes preparations separated from chemical precursors or other chemicals which are involved in the synthesis of the protein, polynucleotide or molecule. In one embodiment, the language “substantially free of chemical precursors or other chemicals” includes preparations of protein having less than about 30% (by dry weight) of chemical precursors or other chemicals, more preferably less than about 20% chemical precursors or other chemicals, still more preferably less than about 10% chemical precursors or other chemicals, and most preferably less than about 5% chemical precursors or other chemicals.

As used herein, a “biologically active portion” of a protein includes a fragment of a protein comprising amino acid sequences sufficiently homologous to, or derived from, the amino acid sequence of the protein, which include fewer amino acids than the full length protein, and exhibits at least one activity of the full-length protein. Typically a biologically active portion comprises a domain or motif with at least one activity of the protein. A biologically active portion of a protein can be a polypeptide which is, for example, 10, 25, 50, 100, 200 or more amino acids in length. In one embodiment, a biologically active portion of a GPCR-signaling protein can be used as a target for developing agents which modulate GPCR-signal transduction.

“Abnormally” expressed, as applied to a gene, includes the abnormal production of mRNA transcribed from a gene or the abnormal production of polypeptide from a gene. An abnormally expressed gene may be overexpressed or underexpressed as compared to the expression level of a normal cell or control cell. In one aspect, abnormal expression refers to a level of expression that differs from normal levels of expression by one standard of deviation. In a preferred aspect, the differential is 2 times higher or lower than the expression level detected in a control sample.

The term “abnormally” expressed also includes nucleotide sequences in a cell or tissue which differ in expression as compared to a normal cell or control cell. In certain embodiments of the invention, the control cell is a GPCR-containing cell from an individual without manifestation of a GPCR-related disease. In certain embodiments, the control cell is a GPCR-containing cell from a tissue not affected by the GPCR-containing disorder. In certain embodiments of the invention, the control cell is a GPCR-containing cell in the presence of agonist. In certain embodiments the control cell is a test cell comprising: i) a GPCR, ii) an RGS, iii) a corresponding Gα protein expressed at a level capable of attenuating GPCR-signaling by at least 50% as compared to a cell without said Gα protein expression, and iv) a reporter gene. In certain embodiments, expression is compared between a GPCR-containing cell or test cell exposed to an agonist or test compound relative to a GPCR-containing cell or test cell which is not exposed to an agonist or test compound. In certain embodiments, expression is compared between a GPCR-containing cell from a tissue not affected by the GPCR-containing disorder with that of an affected tissue. In certain embodiments, the normal cell or control cell or sample is substantially free of a GPCR-related disorder.

As used herein, the term “aberrant” includes gene or protein expression or activity which deviates from the normal expression or activity. Aberrant expression or activity includes increased or decreased expression or activity, as well as expression or activity which does not follow the normal developmental pattern of expression or the subcellular pattern of expression. For example, aberrant expression or activity is intended to include the cases in which a mutation in a gene causes the gene to be under-expressed or over-expressed and situations in which such mutations result in a non-functional protein or a protein which does not function in a normal fashion. In certain embodiments, the normal cell or sample cell or control cell is substantially free of a GPCR-related disorder.

As used herein, the term “modulation” includes, in its various grammatical forms (e.g., “modulated”, “modulation”, “modulating”, etc.), up-regulation, induction, stimulation, potentiation, attenuation, and/or relief of inhibition, as well as inhibition and/or down-regulation or suppression.

A “probe” when used in the context of polynucleotide manipulation includes an oligonucleotide that is provided as a reagent to detect a target present in a sample of interest by hybridizing with the target. Usually, a probe will comprise a label or a means by which a label can be attached, either before or subsequent to the hybridization reaction. Suitable labels include, but are not limited to radioisotopes, fluorochromes, chemiluminescent compounds, dyes, and proteins, including enzymes.

A “primer” includes a short polynucleotide, generally with a free 3′-OH group that binds to a target or “template” present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or “set or primers” consisting of an “upstream” and a “downstream” primer, and a catalyst of polymerization, such as a DNA polymerase, and typically a thermally-stable polymerase enzyme. Methods for PCR are well known in the art, and are taught, for example, in MacPherson et al., IRL Press at Oxford University Press (1991). All processes of producing replicate copies of a polynucleotide, such as PCR or gene cloning, are collectively referred to herein as “replication.” A primer can also be used as a probe in hybridization reactions, such as Southern or Northern blot analyses (see, e.g., Sambrook, Fritsh and Maniatis, Molecular Cloning: A Laboratory Manual. 2nd, ed., Cold Spring Harbor Laboratory, Cold Spring Harbor Laboratory Press, Cold Spring Harbor, N.Y., 1989).

The term “cDNAs” includes DNA that is complementary to mRNA molecules present in a cell or organism mRNA that can be converted into cDNA with an enzyme such as reverse transcriptase. A “cDNA library” includes a collection of mRNA molecules present in a cell or organism, converted into cDNA molecules with the enzyme reverse transcriptase, then inserted into “vectors” (other DNA molecules that can continue to replicate after addition of foreign DNA). Exemplary vectors for libraries include bacteriophage, viruses that infect bacteria (e.g., lambda phage). The library can then be probed for the specific cDNA (and thus mRNA) of interest. Many types of cDNA libraries are commercially available and may be used in connection with the invention.

A “gene delivery vehicle” includes a molecule that is capable of inserting one or more polynucleotides into a host cell. Examples of gene delivery vehicles are liposomes; biocompatible polymers, including natural polymers and synthetic polymers; lipoproteins; polypeptides; polysaccharides; lipopolysaccharides; artificial viral envelopes; metal particles, and bacteria; viruses, viral vectors, such as baculovirus adenovirus, and retrovirus, bacteriophage, cosmid, plasmid, fungal vector and other recombination vehicles typically used in the art which have been described for replication and/or expression in a variety of eukaryotic and prokaryotic hosts. The gene delivery vehicles may be used for replication of the inserted polynucleotide, gene therapy, as well as simply for polypeptide and protein expression.

A “vector” includes a self-replicating nucleic acid molecule that transfers an inserted polynucleotide into and/or between host cells. The term is intended to include vectors that function primarily for insertion of a nucleic acid molecule into a cell, replication vectors that function primarily for the replication of nucleic acid and expression vectors that function for transcription and/or translation of the DNA or RNA. Also intended are vectors that provide more than one of the above function.

A “host cell” is intended to include any individual cell or cell culture which can be, or has been, a recipient for vectors or for the incorporation of exogenous polynucleotides and/or polypeptides. It also is intended to include progeny of a single cell. The progeny may not necessarily be completely identical (in morphology or in genomic or total DNA complement) to the original parent cell due to natural, accidental, or deliberate mutation. The cells may be prokaryotic or eukaryotic, and include but are not limited to bacterial cells, yeast cells, insect cells, animal cells, and mammalian cells, including but not limited to murine, rat, simian or human cells.

The term “genetically modified” includes a cell containing and/or expressing a foreign or exogenous gene or polynucleotide sequence which in turn modifies the genotype or phenotype of the cell or its progeny. “Genetically modified” also includes a cell containing or expressing a gene or polynucleotide sequence which has been introduced into the cell. For example, in this embodiment, a genetically modified cell has had introduced a gene which gene is also endogenous to the cell. The term “genetically modified” also includes any addition, deletion, or disruption to a cell's endogenous nucleotides.

As used herein, “expression” includes the process by which polynucleotides are transcribed into RNA and/or translated into polypeptides. If the polynucleotide is derived from genomic DNA, expression may include splicing of the RNA, if an appropriate eukaryotic host is selected. Regulatory elements required for expression include promoter sequences to bind RNA polymerase and transcription initiation sequences for ribosome binding. For example, a bacterial expression vector includes a promoter such as the lac promoter and for transcription initiation the Shine-Dalgarno sequence and the start codon AUG. Similarly, a eukaryotic expression vector includes a heterologous or homologous promoter for RNA polymerase II, a downstream polyadenylation signal, the start codon AUG, and a termination codon for detachment of the ribosome. Such vectors can be obtained commercially or assembled by the sequences described in methods well known in the art, for example, the methods described below for constructing vectors in general.

As used herein, a “test sample” includes a biological sample obtained from a subject of interest. For example, a test sample can be a biological fluid (e.g., blood, lymph, cerebral-spinal fluid), cell sample, or a tissue sample (e.g., tissue obtained from a biopsy).

As used herein, “hybridization” includes a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Hybridization reactions can be performed under conditions of different “stringency”. The stringency of a hybridization reaction includes the difficulty with which any two nucleic acid molecules will hybridize to one another. The present invention also includes polynucleotides capable of hybridizing under reduced stringency conditions, more preferably stringent conditions, and most preferably highly stringent conditions, to polynucleotides described herein. Examples of stringency conditions are shown in Table 2 below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE 2
STRINGENCY CONDITIONS

| Stringency | Poly-nucleotide | Hybrid Length | Hybridization Temperature | Wash Temperature |
|---|---|---|---|---|
| Condition | Hybrid | (bp)¹ | and Buffer^{H} | and Buffer^{H} |
| A | DNA:DNA | >50 | 65° C.; 1xSSC -or- | 65° C.; 0.3xSSC |
| | | | 42° C.; 1xSSC, 50% formamide | |
| B | DNA:DNA | <50 | T_{B}*; 1xSSC | T_{B}*; 1xSSC |
| C | DNA:RNA | >50 | 67° C.; 1xSSC -or- | 67° C.; 0.3xSSC |
| | | | 45° C.; 1xSSC, 50% formamide | |
| D | DNA:RNA | <50 | T_{D}*, 1xSSC | T_{D}*; 1xSSC |
| E | RNA:RNA | >50 | 70° C.; 1xSSC -or- | 70° C.; 0.3xSSC |
| | | | 50° C.; 1xSSC, 50% formamide | |
| F | RNA:RNA | <50 | T_{F}*; 1xSSC | T_{f}*; 1xSSC |
| G | DNA:DNA | >50 | 65° C.; 4xSSC -or- | 65° C.; 1xSSC |
| | | | 42° C.; 4xSSC, 50% formamide | |
| H | DNA:DNA | <50 | T_{H}*; 4xSSC | T_{H}*; 4xSSC |
| I | DNA:RNA | >50 | 67° C.; 4xSSC -or- | 67° C.; 1xSSC |
| | | | 45° C.; 4xSSC, 50% formamide | |
| J | DNA:RNA | <50 | T_{J}*; 4xSSC | T_{J}*; 4xSSC |
| K | RNA:RNA | >50 | 70° C.; 4xSSC -or- | 67° C.; 1xSSC |
| | | | 50° C.; 4xSSC, 50% formamide | |
| L | RNA:RNA | <50 | T_{L}*; 2xSSC | T_{L}*; 2xSSC |
| M | DNA:DNA | >50 | 50° C.; 4xSSC -or- | 50° C.; 2xSSC |
| | | | 40° C.; 6xSSC, 50% formamide | |
| N | DNA:DNA | <50 | T_{N}*; 6xSSC | T_{N}*; 6xSSC |
| O | DNA:RNA | >50 | 55° C.; 4xSSC -or- | 55° C.; 2xSSC |
| | | | 42° C.; 6xSSC, 50% formamide | |
| P | DNA:RNA | <50 | T_{P}*; 6xSSC | T_{P}*; 6xSSC |
| Q | RNA:RNA | >50 | 60° C.; 4xSSC -or- | 60° C.; 2xSSC |
| | | | 45° C.; 6xSSC, 50% formamide | |
| R | RNA:RNA | <50 | T_{R}*; 4xSSC | T_{R}*; 4xSSC |
| ¹: The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity. | | | | |
| ^{H}: SSPE (1 × SSPE is 0.15 M NaCl, 10 mM NaH₂PO₄, and 1.25 mM EDTA, pH 7.4) can be substituted for SSC (1 × SSC is 0.15 M NaCl and 15 mM sodium citrate) in the hybridization and wash buffers; washes are performed for 15 minutes after hybridization is complete. | | | | |
| T_{B}*-T_{R}*: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# or A ⁺ T bases) ⁺ 4(# of G ⁺ C bases). For hybrids between 18 and 49 base pairs in length, | | | | |
| #T_{m(° C.) = 81.5} ⁺ 16.6(log₁₀Na⁺) ⁺ 0.41(%G⁺C) − (600/N), where N is the number of bases in the hybrid, and Na⁺ is the concentration of sodium ions in the hybridization buffer (Na⁺ for 1 × SSC = 0.165 M). | | | | |

Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, NY, chapters 9 and 11, and Current Protocols in Molecular Biology, 1995, F. M. Ausubel et al,, eds., John Wiley & Sons, Inc., sections 2.10 and 6.3-6.4, incorporated herein by reference.

When hybridization occurs in an antiparallel configuration between two single-stranded polynucleotides, the reaction is called “annealing” and those polynucleotides are described as “complementary”. A double-stranded polynucleotide can be “complementary” or “homologous” to another polynucleotide, if hybridization can occur between one of the strands of the first polynucleotide and the second. “Complementarity” or “homology” (the degree that one polynucleotide is complementary with another) is quantifiable in terms of the proportion of bases in opposing strands that are expected to hydrogen bond with each other, according to generally accepted base-pairing rules.

An “antibody” includes an immunoglobulin molecule capable of binding an epitope present on an antigen. As used herein, the term encompasses not only intact immunoglobulin molecules such as monoclonal and polyclonal antibodies. but also anti-idotypic antibodies, mutants, fragments, fusion proteins, bi-specific antibodies, humanized proteins or antibodies, and modifications of the immunoglobulin molecule that comprises an antigen recognition site of the required specificity.

As used herein, the term “normal” when used in connection with “cell”, “tissue”, or “sample” refers to cells, tissues or other such samples from a subject who has not suffered the GPCR-related disorder, or from a cell, tissue or sample that is substantially free of a GPCR-related disorder. In certain embodiments, control samples of the present invention are taken from normal samples. As used herein, a “control level of expression” refers to the level of expression associated with normal samples or cells.

Various aspects of the invention are described in further detail in the following subsections, which describe in more detail the present invention. The use of “subsections” is not meant to limit the invention as subsections may apply to any aspect of the invention.

GPCR-Signaling

Without limitation as to mechanism, the present invention is based on the discovery that certain Gα proteins can facilitate attenuation of signaling from a GPCR. Gαi and Gαq classes of protein have been discovered to enhance the inhibitory effects of certain RGS proteins. Accordingly, the Gαi or Gαq proteins, in combination with their respective RGS proteins, attenuate GPCR signaling.

Without limitation, the invention is further based on the discovery that the expression level of Gαi or Gαq contributes to the attenuation of signaling.

In a specific embodiment exemplified herein, a GPCR signaling pathway was demonstrated to be attenuated and inhibited by the co-expression of an RGS and Gαi. In the absence of these co-expressed molecules, the GPCR signaling pathway is capable of eliciting a response when a GPCR is contacted by a GPCR agonist. This response can be detected by a number of techniques known in the art. One technique for detecting GPCR-signaling is to provide the GPCR-containing cell with a reporter gene, which is transcribed in response to GPCR signaling. In this embodiment, introduction of an RGS of the invention into the cell lead to an inhibition of GPCR signaling by approximately 30-40% as compared to signaling without the RGS. Surprisingly, co-transfection of the RGS with a corresponding Gα protein led to an inhibition of GPCR signaling by approximately 80-90% as compared to signaling without the RGS or Gα molecules. Accordingly, Gαi or Gαq molecules in the presence of a corresponding RGS are capable of attenuating GPCR-signaling.

This increased attenuation is useful for drug screening because the amplified attenuation facilitates observation of reliable positive and negative results. Accordingly, certain embodiments of the invention provide methods for attenuating GPCR signaling which methods are useful for drug screening assays, diagnostics, prognostics and treatment of GPCR-related disorders.

The attenuation of signaling by Gαi or Gαq, in combination with RGS, further provides methods and compositions useful in treatment of GPCR-related disorders.

In another embodiment, the present invention pertains to the use of RGS and Gα proteins listed in Table 1, polynucleotides, and the encoded polypeptides as GPCR signaling molecules and therapeutic targets for GPCR-related disorders. With respect to such GPCR-related disorders, these signaling molecules are further useful to correlate differences in levels of expression with a poor or favorable prognosis. The RGS proteins and Gα proteins of the invention are also useful in assessing the efficacy of a treatment or therapy of GPCR-related disorders, or as a target for a treatment. The invention further provides methods for inhibiting GPCR-related disorders, and methods for identifying RGS inhibitors which are useful in the treatment of GPCR-related disorders.

Therefore, without limitation as to mechanism, the invention is based in part on the principle that certain RGS proteins in combination with certain Gα proteins of the invention attenuate GPCR signaling and may ameliorate GPCR-related disorders when expressed at levels similar to, or substantially similar to, normal (non-diseased) cells. Further, the invention is based in part on the principle that certain RGS proteins in combination with certain Gα proteins of the invention attenuate GPCR signaling and may ameliorate GRCR-related disorders when active at a level similar to, or substantially similar to, normal (non-diseased) cells. Still further, the invention is based in part on the principle that RGS proteins act, in part, to facilitate the hydrolysis of GTP-bound-Gα to GDP-bound-Gα.

In one aspect, the invention provides RGS and Gα molecules whose level of expression, or activity, is correlated with the presence of a GPCR-related disorder. The RGS molecules and Gα molecules of the invention may be polynucleotides (e.g., DNA; cDNA or mRNA) or peptide(s) or polypeptides. In certain preferred embodiments; the invention is performed by detecting the presence of a transcribed polynucieotide or a portion thereof. Alternatively, detection may be performed by detecting the presence of a protein.

In another aspect of the invention, the expression levels of the RGS and Gα proteins are determined in a particular subject sample for which either diagnosis or prognosis information is desired. In certain embodiments, comparison of relative levels of expression is indicative of the severity of a GPCR-related disorder, and as such permits for diagnostic and prognostic analysis. Moreover, by comparing relative GPCR signaling of a GPCR-related disorder from tissue samples taken at different points in time, e.g., pre- and post-therapy and/or at different time points within a course of therapy, information regarding which genes are important in each of these stages is obtained. One of the skill in the art will recognize other controls such as by using different time points, or the presence or absence of a test compound. One of ordinary skill in the art will appreciate that other post-activation time points may be used to access expression levels of RGS proteins and Gα proteins. For example, post-activation time points include but are not limited to 6 h, 8 h, 12 h, 15 h, 20 h, 24 h, 36 h, 48 h, 72 hours. One skilled in the art will be cognizant of the fact that a preferred detection methodology is one in which the resulting detection values are above the minimum detection limit of the methodology.

The identification of RGS and Gα molecules that are abnormally expressed in a GPCR-related disorder versus normal tissue allows the use of this invention in a number of ways. For example, comparison of expression of RGS and Gα at various disease progression states provides a method for long term prognosing, including survival. In another embodiment, the evaluation of a particular treatment regime may be evaluated, including whether a particular drug will act to improve the long-term prognosis in a particular patient. In this embodiment, the expression and activity of the RGS and Gα molecules of the invention may be correlated with long-term prognosis of a patient.

The discovery of attenuated GPCR-signaling allows for screening of test compounds with an eye to modulating a particular signaling pattern; for example, screening can be done for compounds that will convert a signaling profile for a poor prognosis to a better prognosis. These methods can also be done on the protein level; that is; protein expression levels of RGS proteins in GPCR-related disorders can be evaluated for diagnostic and prognostic purposes or to screen test compounds. For example, in relation to these embodiments, the RGS or Gα molecules of the invention may have modulated activity or expression in response to a therapy regime. Alternatively, the modulation of the activity or expression of such molecules may be correlated with the diagnosis or prognosis of a GPCR-related disorder. In addition, RGS and Gα molecules can be administered for gene therapy purposes. For example, antisense oligonucleotides corresponding to RGS or Gα proteins may be administered to decrease the expression or activity of these proteins. Such administration can led to increased GPCR-signaling and amelioration of GPCR-related disorders.

In another embodiment of the invention, one of more GPCR-signaling molecules can be used as a therapeutic compound of the invention. In yet other embodiments, an inhibitor of an RGS of the invention may be used as a therapeutic compound of the invention, or may be used in combination with one or more other therapeutic compositions of the invention. Formulation of such compounds into pharmaceutical compositions is described in subsections below.

Sources of Markers

The polynucleotides and polypeptides comprising an RGS or Gαi or Gαq of the invention or active portion thereof, may be isolated from any tissue or cell of a subject, or, alternatively, may be synthesized by techniques known in the art. In a preferred embodiment, the tissue is from the nervous system or cardiovascular system. However, it will be apparent to one skilled in the art that tissue samples, including bodily fluids such as blood, may also serve as sources from which the RGS or Gα molecules of the invention may be assessed. The tissue samples containing one or more of the RGS or Gα molecules of the invention themselves may be useful in the methods of the invention, and one skilled in the art will be cognizant of the methods by which such samples may be conveniently obtained, stored and/or preserved.

Isolated Polynucleotides

One aspect of the invention pertains to isolated polynucleotide (e.g., DNA, cDNA, mRNA) molecules comprising the RGS and Gα molecules of the invention, or polynucleotides which encode the polypeptide molecules of the invention, or fragments thereof. Another aspect of the invention pertains to isolated polynucleotide fragments sufficient for use as hybridization probes to identify the polynucleotide molecules encoding the markers for the invention in a sample, as well as nucleotide fragments for use as PCR primers of the amplification or mutation of the nucleic acid molecules which encode the GPCR-signaling molecules of the invention. Another aspect of the invention pertains to isolated RGS and Gα polynucleotides of the invention for use in gene therapy, such as antisense and ribozyme therapies.

A polynucleotide molecule of the present invention, or homolog thereof, or a portion thereof, can be isolated using standard molecular biology techniques and the sequence information known in the art. Using all or portions of the polynucleotide sequence of one of the RGS or Gα molecules listed in Table 1 (or a homolog thereof as a hybridization probe, a marker gene of the invention or a polynucleotide molecule encoding a marker polypeptide of the invention can be isolated using standard hybridization and cloning techniques (e.g., as described in Sambrook, Fritsh and Maniatis, Molecular Cloning: A Laboratory Manual 2nd, ed., Cold Spring Harbor Laboratory, Cold Spring Harbor Laboratory Press, Cold spring Harbor, N.Y., 1989).

A polynucleotide of the invention can be amplified using cDNA, mRNA or alternatively, genomic DNA, as a template and appropriate oligonucleotide primers according to standard PCR amplification techniques. The polynucleotide so amplified can be cloned into an appropriate vector and characterized by DNA sequence analysis. Furthermore, oligonucleotides corresponding to RGS or Gα polynucleotides of the invention sequences, or nucleotide sequences encoding a polypeptide of the invention, can also be prepared by standard synthetic techniques, e.g., using an automated DNA synthesizer.

In a preferred embodiment, an isolated polynucleotide of the invention comprises a polynucleotide molecule which is a complement of the nucleotide sequence of a RGS or Gα polynucleotide of the invention, or homolog thereof, or a portion of any of these nucleotide sequences. A polynucleotide which is complementary to such a nucleotide sequence is one which is sufficiently complementary to the nucleotide sequence such that it can hybridize to the nucleotide sequence, thereby forming a stable duplex. In a preferred embodiment, the complementary nucleotide sequence is capable of hybridizing to the target nucleotide sequence under conditions of high stringency.

The polynucleotide molecules of the invention, moreover, can comprise only a portion of the polynucleotide sequence of an RGS or Gα polynucleotide of the invention, or a gene encoding an RGS or Gα polypeptide of the invention, for example, a fragment which can be used as a probe or primer. The probe/primer typically comprises substantially purified oligonucleotide. The oligonucleotide typically comprises a region of nucleotide sequence that hybridizes under stringent conditions to at least about 7 or 15, preferably about 20 or 25, more preferably about 50, 75, 100, 125, 150, 175, 200, 225, 250, 275, 300, 325, 350, 400 or more consecutive nucleotides of the RGS or Gα polynucleotide of the invention.

Probes based on the nucleotide sequence of a marker gene or of a polynucleotide molecule encoding a marker polypeptide of the invention can be used to detect transcripts or genomic sequences corresponding to the marker gene(s) and/or marker polypeptide(s) of the invention. In preferred embodiments, the probe comprises a label group attached thereto, e.g., the label group can be a radioisotope, a fluorescent compound, an enzyme, or an enzyme co-factor. Such probes can be used as a part of a diagnostic test kit for identifying cells or tissue which misexpress (e.g., over- or under-express) a marker polynucleotide or polypeptide of the invention, or which have greater or fewer copies of an RGS or Gα gene of the invention. For example, a level of a RGS or Gα molecule of the invention in a sample of cells from a subject may be detected, the amount of polypeptide or mRNA transcript of a gene encoding the RGS or Gα polypeptide may be determined, or the presence of mutations or deletions of a marker gene of the invention may be assessed.

Homologs, Allelic Variants and Mutants

The invention also specifically encompasses homologs of the RGS and Gα molecules of the invention, particularly human homologs. Gene homologs are well understood in the art and are available using databases or search engines such as the Pubmed-Entrez database.

The invention further encompasses polynucleotide molecules that, because of the degeneracy of the genetic code, encode the same proteins as shown in Table 1.

The invention also encompasses polynucleotide molecules which are structurally different from the molecules described above (i.e. which have a slight altered sequence), but which have substantially the same properties as the molecules above (e.g., encoded amino acid sequences, or which are changed only in nonessential amino acid residues). Such molecules include allelic variants and are described in greater detail in subsections herein.

In addition to the nucleotide sequences of the RGS proteins and Gα proteins of the invention (which may be known in the art, as disclosed in U.S. Pat. No. 6,069,296 and U.S. Pat. No. 5,929,207), it will be appreciated by those skilled in the art that DNA sequence polymorphisms that lead to changes in the amino acid sequences of the proteins listed in Table 1 may exist within a population (e.g., the human population). Such genetic polymorphism in the proteins listed in Table 1 may exist among individuals within a population due to natural allelic variation. An allele is one of a group of genes which occur alternatively at a given genetic locus. In addition, it will be appreciated that DNA polymorphisms that affect RNA expression levels can also exist that may affect the overall expression level of that gene (e.g., by affecting regulation or degradation). As used herein, the phrase “allelic variant” includes a nucleotide sequence which occurs at a given locus or to a polypeptide encoded by the nucleotide sequence.

In another embodiment, an isolated polynucleotide molecule of the invention is at least 15, 20, 25, 30, 50, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000 or more nucleotides in length and hybridizes under stringent conditions to a RGS or Gα polynucleotide molecule corresponding to a RGS or Gα protein of the invention. In certain embodiments, the hybridization under stringent conditions is intended to describe conditions for hybridization and washing under which nucleotide sequences at least 60% homologous to each other typically remain hybridized to each other. Preferably, the conditions are such that sequences at least about 70%, more preferably at least about 80%, even more preferably at least about 85% or 90% homologous to each other, typically remain hybridized to each other. Such stringent conditions are known to those skilled in the art and can be found in Current Protocols in Molecular Biology; John Wiley & Sons, N.Y. (1989), 6.3.1-6.3.6.

In addition to naturally-occurring allelic variants of the genes encoding a RGS or Gα protein of the invention that may exist in the population, the skilled artisan will further appreciate that changes can be introduced by mutation into the nucleotide sequences of the genes or polynucleotides of the invention, thereby leading to changes in the amino acid sequence of the encoded proteins, without altering the functional activity of these proteins. For example, nucleotide substitutions leading to amino acid substitutions at “non-essential” amino acid residues can be made. A “non-essential” amino acid residue is a residue that can be altered from the wild-type sequence of a protein without altering the biological activity, whereas an “essential” amino acid residue is required for biological activity. For example, amino acid residues that are conserved among allelic variants (i.e., “essential”) or homologs of a gene (e.g., among homologs of a gene from different species) are predicted to be particularly unamenable to alteration.

In yet other aspects of the invention, polynucleotides of a RGS or Gα molecule may comprise one or more mutations. An isolated polynucleotide molecule encoding a protein with a mutation in a RGS or Gα protein of the invention can be created by introducing one or more nucleotide substitutions, additions or deletions into the nucleotide sequence of the gene encoding the marker protein, such that one or more amino acid substitutions, additions or deletions are introduced into the encoded protein. Such techniques are well known in the art. Mutations can be introduced into the polynucleotides of the invention by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine) Alternatively mutations can be introduced randomly along all or part of a coding sequence of a RGS or Gα gene of the invention, such as by saturation mutagenesis, and the resultant mutants can be screened for biological activity to identify mutants that retain activity. Following mutagenesis, the encoded protein can be expressed recombinantly and the activity of the protein can be determined.

In other embodiments, an oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Pros. Natl. Acad Sci. USA 84:648-652; PCT Publication No. WO88/09810) or the blood-kidney barrier (see, e.g., PCT Publication No. WO89/10134). In addition, oligonucleotides can be modified with hybridization-triggered cleavage agents (See, e.g., Krol et al. (1988) Bio-Techniques 6:958-976) or intercalating agents. (See, e.g., Zon (1988) Pharm. Res. 5:539-549). To this end, the oligonucleotide may be conjugated to another molecule (e.g., a peptide, hybridization triggered cross-linking agent, transport agent, or hybridization-triggered cleavage agent). Finally, the oligonucleotide may be detectably labeled, either such that the label is detected by the addition of another reagent (e.g., a substrate for an enzymatic label), or is detectable immediately upon hybridization of the nucleotide (e.g., a radioactive label, fluorescent label, or a molecular beacon, as described in U.S. Pat. No. 5,876,930).

Antisense and Ribozyme Molecules

Another aspect of the invention pertains to isolated polynucleotide molecules which are antisense to the RGS or Gα polynucleotides of the invention. An “antisense” polynucleotide comprises a nucleotide sequence which is complementary to a “sense” polynucleotide encoding a protein, e.g., complementary to the coding strand of a double-stranded cDNA molecule or complementary to an mRNA sequence. Accordingly, an antisense polynucleotide can hydrogen bond to a sense polynucleotide. The antisense polynucleotide can be complementary to an entire coding strand of a gene of the invention or to only a portion thereof. In one embodiment, an antisense polynucleotide molecule is antisense to a “coding region” of the coding strand of a nucleotide sequence of the invention. The term “coding region” includes the region of the nucleotide sequence comprising codons which are translated into amino acid. In another embodiment, the antisense polynucleotide molecule is antisense to a “noncoding region” of the coding strand of a nucleotide sequence of the invention.

Antisense polynucleotides of the invention can be designed according to the rules of Watson and Crick base pairing. The antisense polynucleotide molecule can be complementary to the entire coding region of an mRNA corresponding to a gene of the invention, but more preferably is an oligonucleotide which is antisense to only a portion of the coding or noncoding region. An antisense oligonucleotide can be. for example, about 5, 10, 15, 20, 25, 30, 35, 40, 45 or 50 nucleotides in length. For example, an antisense RGS may be preferably an oligonucleotide which is antisense to a portion of the RGS core domain.

An antisense polynucleotide of the invention can be constructed using chemical synthesis and enzymatic ligation reactions using procedures known in the art. For example, an antisense polynucleotide (e.g., an antisense oligonucleotide) can be chemically synthesized using naturally occurring nucleotides or variously modified nucleotides designed to increase the biological stability of the molecules or to increase the physical stability of the duplex formed between the antisense and sense polynucleotides, e.g., phosphorothioate derivatives and acridine substituted nucleotides can be used. Examples of modified nucleotides which can be used to generate the antisense polynucleotide include 5-fluorouracil, 5-bromouracil, 5-chlorouracil, 5-iodouracil, hypoxanthine, xantine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladen4exine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, an antisense polynucleotide can be produced biologically using an expression vector into which a polynucleotide has been subcloned in an antisense orientation (i.e., RNA transcribed from the inserted polynucleotide will be of an antisense orientation to a target polynucieotide of interest, described further in the following subsection).

The antisense polynucleotide molecules of the invention are typically administered to a subject or generated in situ such that they hybridize with or bind to cellular mRNA and/or genomic DNA encoding an RGS or Gα protein of the invention to thereby inhibit expression of the protein, e.g., by inhibiting transcription and/or translation. The hybridization can be by conventional nucleotide complementarity to form a stable duplex, or, for example, in the cases of an antisense polynucleotide molecule which binds to DNA duplexes, through specific interactions in the major groove of the double helix. An example of a route of administration of antisense polynucleotide molecules of the invention include direct injection at a tissue site (e.g., lymph node, heart, or blood). Alternatively, antisense polynucleotide molecules can be modified to target selected cells and then administered systemically. For example, for systemic administration, antisense molecules can be modified such that they specifically bind to receptors or antigens expressed on a selected cell surface, e.g., by linking the antisense polynucleotide molecules to peptides or antibodies which bind to cell surface receptors or antigens. In certain embodiments of the invention, it is advantageous, for example when treating a neuropsychiatric disorder, to target neuronal or brain cells. In such embodiments, neuronal-specific antigens include, but are not limited to, dopamine receptors, serotonin receptors, serotonin transporters, M2 receptors, 5HTIA receptors, Edg1 receptors and Bradykinin receptors. The antisense polynucleotide molecules can also be delivered to cells using the vectors described herein or known in the art. To achieve sufficient intracellular concentrations of the antisense molecules, vector constructs in which the antisense polynucleotide molecule is placed under the control of a strong pol II or pol III promoter are preferred.

In yet another embodiment, the antisense polynucleotide molecule of the invention is an α-anomeric polynucleotide molecule. An α-anomeric polynucleotide molecule forms specific double-stranded hybrids with complementary RNA in which, contrary to the usual β-units, the strands run parallel to each other (Gaultier et al. (1987) Polynucieotides. Res. 15:6625-6641). The antisense polynucleotide molecule can also comprise a 2′-o-methylribonucleotide (Inoue et al. (1987) Polynucleotides Res. 15:6131-6148) or a chimeric RNA-DNA analogue (Inoue et al (1987) FEBS Lett. 215:327-330).

In still another embodiment, an antisense polynucleotide of the invention is a ribozyme. Ribozymes are catalytic RNA molecules with ribonuclease activity which are capable of cleaving a single-stranded polynucleotide, such as an mRNA, to which they have a complementary region. Thus, ribozymes (e.g., hammerhead ribozymes (described in Haselhoif and Gerlach (1988) Nature 334:585-591)) can be used to catalytically cleave mRNA transcripts of the marker genes of the invention to thereby inhibit translation of said mRNA. A ribozyme having specificity for a RGS or Gα polynucleotide can be designed based upon the nucleotide sequence of a gene of the invention, disclosed herein. For example, a derivative of a Tetrahymena L-19 IVS RNA can be constructed in which the nucleotide sequence of the active site is complementary to the nucleotide sequence to be cleaved in a marker protein-encoding mRNA. See, e.g., Cech et al. U.S. Pat. No. 4,987,071; and Cech et al. U.S. Pat. No. 5,116,742. Alternatively, mRNA transcribed from a gene of the invention can be used to select a catalytic RNA having a specific ribonuclease activity from a pool of RNA molecules. See, e.g., Bartel, D. and Szostak, J. W. (1993) Science 261:1411-1418.

Alternatively, expression of a RGS or Gα gene of the invention can be inhibited by targeting nucleotide sequences complementary to the regulatory region of these genes (e.g., the promoter and/or enhancers) to form triple helical structures that prevent transcription of the gene in target cells. See generally, Helene, C. (1991) Anticancer Drug Des. 6(6):569-84; Helene, C. et al. (1992) Ann. N.Y. Acad Sci. 660:27-36; and Maher, L. J. (1992) Bioassays 14(12):807-15.

Expression of the RGS and Gα genes and proteins of the invention, can also be inhibited using RNA interference (“RNAₗ”). This is a technique for post transcriptional gene silencing (“PTGS”), in which target gene activity is specifically abolished with cognate double-stranded RNA (“dsRNA”). RNAᵢ resembles in many aspects PTGS in plants and has been detected in many invertebrates including trypanosome, hydra, planaria, nematode and fruit fly (Drosophila melanogaster). It may be involved in the modulation of transposable element mobilization and antiviral state formation. RNAₗ in mammalian systems is disclosed in PCT application WO 00/63364. which is incorporated by reference herein in its entirety Generally, dsRNA of at least about 21 nucleotides; homologous to the target gene, is introduced into the cell and a sequence specific reduction in gene activity is observed. See e.g., Elbashir et al., (2001) Nature 6836:494-498.

In yet another embodiment, the polynucleotide molecules of the present invention can be modified at the base moiety, sugar moiety or phosphate backbone to improve, e.g., the stability, hybridization, or solubility of the molecule. For example, the deoxyribose phosphate backbone of the polynucleotide molecules can be modified to generate peptide polynucleotides (see Hyrup B. et al. (1996) Bioorganic & Medicinal Chemistry 4(1): 523). As used herein, the terms “peptide polynucleotides” or “PNAs” refer to polynucleotide mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al., (1996) supra; Perry-O'Keefe et al., Proc. Natl. Acad. Sci. 93: 14670-675.

PNAs can be used in therapeutic and diagnostic applications. For example, PNAs can be used as antisense or antigene agents for sequence-specific modulation of marker gene expression by, for example, inducing transcription or translation arrest or inhibiting replication. PNAs of the RGS or Gα polynucleotide molecules of the invention, or homologs thereof, can also be used in the analysis of single base pair mutations in a gene, (e.g., by PNA-directed PCR clamping); as “artificial restriction enzymes” when used in combination with other enzymes, (e.g., S1 nucleases (Hyrup (1996) supra); or as probes or primers for DNA sequencing or hybridization (Hyrup (1996) supra; Perry-O'Keefe supra).

In another embodiment, PNAs can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of the polynucleotide molecules of the invention can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. (1996) supra). The synthesis of PNA-DNA chimeras can be performed as described in Hyrup B. (1996) supra and Finn P. J. et al. (1996) Polynucleotides Res. 24 (17): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a spacer between the PNA and the 5′ end of DNA (Mag, M. et al. (1989) Polynucleotide Res. 17: 5973-88). PNA monomers are then coupled in a stepwise manner to produce a chimeric molecule with a 5′ PNA segment and a 3′ DNA segment (Finn P. J. et al. (1996) supra). Alternatively, chimeric molecules can be synthesized with a 5′ DNA segment and a 3′ PNA segment (Peterser, K. H. et al. (1975) Bioorganic Med Chem. Lett. 5: 1119-11124).

Isolated Polypeptides

Several aspects of the invention pertain to isolated RGS and Gα proteins, and biologically active portions thereof, as well as polypeptide fragments suitable for use as immunogens to raise anti-marker protein antibodies. In one embodiment, native marker proteins can be isolated from cells or tissue sources by an appropriate purification scheme using standard protein purification techniques. In another embodiment, RGS or Gα proteins of the invention are produced by recombinant DNA techniques. Alternative to recombinant expression, a protein or polypeptide can be synthesized chemically using standard peptide synthesis techniques.

Homologs

The invention provides the use of RGS and Gα proteins set forth in Table 1, or homologs thereof, including human homologs. In other embodiments, the protein is substantially homologous to a protein listed in Table 1, and retains at least one functional activity of the RGS or Gα protein, yet differs in amino acid sequence due to natural allelic variation of the marker gene or mutagenesis, as described in detail above. Accordingly, in another embodiment, the RGS or Gα protein of the invention is a protein which comprises an amino acid sequence at least about 60%, 65%, 70%, 75%, 80%; 85%; 90%; 95%, 98% or more homologous to the amino acid sequence of a RGS or Gα molecule, particularly the RGS proteins listed in Table 1.

To determine the percent identity of two amino acid sequences or of two polynucleotide sequences, the sequences are aligned for optimal comparison purposes (e.g., gaps can be introduced in one or both of a first and a second amino acid or polynucleotide sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or polynucleotide identity is equivalent to amino acid or polynucleotide “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. (48):444-453 (1970)) algorithm, which has been incorporated into the GAP program in the GCG software package, using either a Blossom 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software, using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (CABIOS, 4:11-17 (1989)) which has been incorporated into the ALIGN program (version 2.0), using a PAM 120 weight residue table, a gap length penalty of 12 and a gap penalty of 4.

The polynucleotide and protein sequences of the present invention can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to polynucleotide molecules of the invention. BLAST protein searches can be performed with the XBLAST program, score=50, wordlength=3 to obtain amino acid sequences homologous to the RGS or Gα molecules of the invention. To obtain gapped alignments for comparison purposes, Gapped BLAST can be utilized as described in Altschul et al., (1997) Polynucleotides Res. 25(17):3389-3402. When utilizing BLAST and Gapped BLAST programs, the default parameters of the respective programs (e.g., XBLAST and NBLAST) can be used.

Chimeric Proteins

The invention provides chimeric or fusion proteins of the RGS or Gα proteins of the invention. The polypeptide of a chimeric protein can correspond to all or a portion of a RGS or Gα protein. The invention also provides polynucleotides encoding chimeric proteins. In one preferred embodiment, a chimeric protein comprises at least one biologically active portion of a Gα protein. Within the chimeric protein, the term “operatively linked” is intended to indicate that the first polypeptide and the second or additional polypeptides are fused in-frame to each other. The second or additional polypeptides can be fused to the N-terminus or C-terminus of the first polypeptide. In a preferred embodiment the invention provides a Gα chimeric protein comprising i) a portion of a first Gα protein which is capable of contacting an RGS and ii) a portion of a second Gα protein which is capable of contacting a GPCR. In a specific embodiment, the invention provides a Gαq1i chimeric protein wherein the Gαq protein is capable of contacting RGS or capable of transducing a downstream signal and the Gαi portion of the chimeric is capable of coupling to a GPCR. In a further specific embodiment, the GPCR is D2R (dopamine 2 receptor).

For example, in another specific embodiment, the chimera protein is a fusion protein that possesses all the structural motifs of Gαq except the last 5 amino acids, which are replaced with the last 5 amino acids of Gαi1.

The chimeric proteins of the invention can be incorporated into pharmaceutical compositions and administered to a subject in vivo, as described herein. The chimeric proteins can be used to create corresponding Gα proteins. For example, chimeric Gα proteins can be engineered to be coupled to any GPCR of interest by replacing the natural GPCR-binding site with that of the GPCR binding site of interest.

Moreover, the chimeric proteins of the invention may be engineered to be used as immunogens to produce anti-RGS or anti-Gα antibodies in a subject, to purify RGS binding proteins or in screening assays to identify molecules which inhibit the interaction of an RGS protein with a Gα protein.

Preferably, a chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the chimeric gene can be synthesized by conventional techniques, including automated DNA synthesizers. Alternatively, PCR amplification of gene fragments can be carried out using anchor primers. These anchor primer give rise to complementary overhangs between two consecutive gene fragments which can subsequently be annealed and reamplified to generate a chimeric gene sequence (see, for example, Current Protocols In Molecular Biology, eds. Ausubel et al. John Wiley & Sons: 1992). Moreover, many expression vectors are commercially available that already encode a fusion moiety (e.g., a GST polypeptide). RGS or Gα polynucleotides can be cloned into such an expression vector such that the fusion moiety is linked in-frame to the second or additional protein.

Antibodies

In another aspect, the invention includes antibodies that are specific to proteins corresponding to the markers of the invention. Preferably the antibodies are monoclonal, and most preferably,. the antibodies are humanized, as per the description of antibodies described below.

The invention provides methods of making an isolated hybridoma which produces an antibody useful for diagnosing a patient or animal with a GPCR-related disorder. In this method, a protein corresponding to a RGS or Gα protein of the invention is isolated (e.g., by purification from a cell in which it is expressed or by transcription and translation of a polynucleotide encoding the protein in vivo or in vitro using known methods). A vertebrate, preferably a mammal, such as a mouse, rabbit or sheep, is immunized using the isolated protein or protein fragment. The vertebrate may optionally (and preferably) be immunized at least one additional time with the isolated protein or protein fragment, so that the vertebrate exhibits a robust immune response to the protein or protein fragment. Splenocytes are isolated from the immunized vertebrate and fused with an immortalized cell line to form hybridomas, using any of a variety of methods well known in the art. Hybridomas formed in this manner are then screened using standard methods to identify one or more hybridomas which produce an antibody that specifically binds with the protein or protein fragment. The invention also includes hybridomas made by this method and antibodies made using such hybridomas.

An isolated RGS or Gα protein, or a portion or fragment thereof, can be used as an immunogen to generate antibodies that bind marker proteins using standard techniques for polyclonal and monoclonal antibody preparation. A full-length marker protein can be used or, alternatively, the invention provides antigenic peptide fragments of these proteins for use as immunogens. The antigenic peptide of a RGS or Gα protein comprises at least 8 amino acid residues of an amino acid sequence of a protein set forth in Table 1, and encompasses an epitope of an RGS or Gα protein such that an antibody raised against the peptide forms a specific immune complex with the protein. Preferably, the antigenic peptide comprises at least 10 amino acid residues, more preferably at least 15 amino acid residues, even more preferably at least 20 amino acid residues, and most preferably at least 30 amino acid residues.

Preferred epitopes encompassed by the antigenic peptide are regions of the protein that are located on the surface of the protein, e.g., hydrophilic regions, as well as regions with high antigenicity.

A protein immunogen typically is used to prepare antibodies by immunizing a suitable subject, (e.g., rabbit, goat, mouse or other mammal) with the immunogen An appropriate immunogenic preparation can contain, for example, recombinantly expressed RGS protein or a chemically synthesized RGS polypeptide. The preparation can further include an adjuvant, such as Freund's complete or incomplete adjuvant, or similar immunostimulatory agent. Immunization of a suitable subject with an immunogenic protein preparation induces a polyclonal anti-marker protein antibody response. Techniques for preparing, isolating and using antibodies are well known in the art. (see generally D. Lane and E. Harlow in Antibodies: A Laboratory Manual, Cold Spring Harbor Laboratory Press, New York (1990)).

Accordingly, another aspect of the invention pertains to monoclonal or polyclonal antibodies reactive to RGS or Gα proteins of the invention. Examples of immunologically active portions of immunoglobulin molecules include F(ab) and F(ab′)₂ fragments, which can be generated by treating the antibody with an enzyme such as pepsin. The invention provides polyclonal and monoclonal antibodies that bind to RGS proteins. The invention provides polyclonal and monoclonal antibodies that bind to Gα proteins of the invention (e.g., Gαi or Gαq). In specific embodiments of the invention anitbodies of the invention bind to either Gα₁, Gα₂, Gα₃, Gαz, Gαo or Gαq. In other specific embodiments, antibodies of the invention bind to either RGS2, RGS4 or RGSz1. The term “monoclonal antibody” or “monoclonal antibody composition”, as used herein, includes a population of antibody molecules that contain only one species of an antigen binding site capable of immunoreacting with a particular epitope. A monoclonal antibody composition thus typically displays a single binding affinity for a particular protein with which it immunoreacts.

Polyclonal antibodies can be prepared as described above by immunizing a suitable subject with a protein of interest of the invention. The antibody titer in the immunized subject can be monitored over time by standard techniques, such as with an enzyme linked immunosorbent assay (ELISA) using immobilized protein. If desired, the antibody molecules directed against proteins of interest can be isolated from the mammal (e.g., from the blood) and further purified by well known techniques, such as protein A chromatography, to obtain the IgG fraction. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein (1975) Nature 256-495-497) (see also, Brown et al. (1981) J. immunol. 127:539-46; Brown et al. (1980) J. Biol. Chem. 255:4980-83; Yeh et al. (1976) Proc. Natl. Acad, Sci. USA 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner (1981) Yale J. Biol. Med., 54:387-402; M. L. Gefter et al. (1977) Somatic Cell Genet. 3:231-36). Briefly, an immortal cell line (typically a myeloma) is fused to lymphocytes (typically splenocytes) from a mammal immunized with a protein immunogen as described above, and the culture supernatants of the resulting hybridoma cells are screened to identify a hybridoma producing a monoclonal antibody that binds to a protein of interest.

Any of the many well known protocols used for fusing lymphocytes and immortalized cell lines can be applied for the purpose of generating a monoclonal antibody (see, e.g., G. Galfre et al. (1977) Nature 266:SSOS2; Gefter et al. Somatic Cell Genet., cited supra; Letter, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp210-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed) Hybridoma cells producing a monoclonal antibody of the invention are detected by screening the hybridoma culture supernatants for antibodies that bind to the protein of interest, e.g., using a standard ELISA assay.

Alternative to preparing monoclonal antibody-secreting hybridomas, a monoclonal antibody can be identified and isolated by screening a recombinant combinatorial immunoglobulin library (e.g., an antibody phase display library) with a protein of interest to thereby isolate immunoglobulin library members that bind to the protein of interest. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Fuchs et al. (1991) Bio/Technology 9:1370-1372; Hay et al. (1992) Hum. Antibod Hybridomas 3:81-85; Huse et al. (1989) Science 246:1275-1281; Griffiths et al. (1993) EMBO J 12:725-734; and McCafferty et al. Nature (1990) 348:552-554.

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Cabilly et al. U.S. Pat. No. 4,816,567; Better et al. (1988) Science 240:1041-1043; Liu et al. (1987) Proc. Natl. Acad Sci. USA 84:3439-3443; Liu et al. (1987) J. Immunol. 139:3521 3526;Verhoeyan et al. (1988) Science 239:1534; and Beidler et al. (1988) J. Immunol. 141:4053-4060.

Humanized antibodies are particularly desirable for therapeutic treatment of human subjects. Humanized forms of non-human (e.g. murine) antibodies are chimeric molecules of immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues forming a complementary determining region (CDR) of the recipient are replaced by residues from a CDR of a non-human species (donor antibody), such as mouse, rat or rabbit, having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all, or substantially all, of the CDR regions correspond to those of a non-human immunoglobulin and all or substantially all of the constant regions being those of a human immunoglobulin consensus sequence. The humanized antibody will preferably also comprise at least a portion of an immunoglobulin constant region (Fc), typically that of a human immunoglobulin (Jones et al. Nature 321: 522-525 (1986); Riechmann et al, Nature 323: 323-329 (1988); and Presta Curr. Op. Struct. Biol. 2: 594-596 (1992)).

Such humanized antibodies can be produced using transgenic mice which are incapable of expressing endogenous immunoglobulin heavy and light chain genes, but which can express human heavy and light chain genes. The transgenic mice are immunized in the normal fashion with a selected antigen, e.g., all or a portion of a polypeptide corresponding to a marker of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing humanized antibodies, see Lonberg and Huszar (1995) Int. Rev. Immunol. 13:65-93. For a detailed discussion of this technology for producing humanized antibodies and humanized monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.), can be engaged to provide humanized antibodies directed against a selected antigen using technology similar to that described above.

Humanized antibodies which recognize a selected epitope can be generated using a technique referred to as “guided selection.” In this approach a selected non-human monoclonal antibody, e.g., a murine antibody, is used to guide the selection of a humanized antibody recognizing the same epitope (Jespers et al., 1994, Bio technology 12:899-903).

Commercially available anti-marker antibodies may also be used in the methods of the invention. For example, anti-RGS1, anti-RGS2, anti-RGS3 and anti-Gα antibodies are available from Santa Cruz Biotechnology, Inc, Santa Cruz, Calif. Anti-Gα antibodies are also available from Calbiochem-Novabiochem Corp.

An anti-marker protein antibody can be used to isolate a marker protein of the invention by standard techniques, such as affinity chromatography or immunoprecipitation. An antibody to an RGS or Gα can facilitate the purification of natural proteins from cells and of recombinantly produced proteins expressed in host cells. Moreover, an RGS or Gα antibody can be used to detect a RGS or Gα protein respectively (e.g., in a cellular lysate or cell supernatant on the cell surface) in order to evaluate the abundance and pattern of expression of the protein. Such antibodies can be used diagnostically to monitor protein levels in tissue as part of a clinical testing procedure, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatasc, galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Recombinant Expression Vectors and Host Cells

Another aspect of the invention pertains to vectors, preferably expression vectors, containing a polynucleotide encoding a RGS or Gα molecule of the invention or a portion thereof. As used herein, the term “vector” includes a polynucleotide, molecule capable of transporting another polynucleotide to which it has been linked. One type of vector is a “plasmid”, which includes a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors host cell (e.g., replication defective retroviruses, adenoviruses and adeno-associated viruses), which serve equivalent functions.

The recombinant expression vectors of the invention comprise a polynucleotide of the invention in a form suitable for expression of the polynucleotide in a host cell, which means that the recombinant expression vectors include one or more regulatory sequences, selected on the basis of the host cells to be used for expression, which is operatively linked to the polynucleotide sequence to be expressed. Within a recombinant expression vector, “operably linked” is intended to mean that the nucleotide sequence of interest is linked to the regulatory sequences in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology Methods in Enzymology 185, Academic Press, San Diego, Calif. (1990). Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by polynucleotides as described herein (e.g., RGS or Gαi or Gαq proteins, mutant forms of such proteins, chimeric proteins, and the like).

The recombinant expression vectors of the invention can be designed for expression of proteins or polynucleotides in prokaryotic or eukaryotic cells. In specific embodiments of the invention, RGS2, RGS4 and RGSz1 were cloned into the eukaryotic expression vector pCR31. For example, a protein of interest can be expressed in bacterial cells such as E. coli, insect cells (using baculovirus expression vectors) yeast cells or mammalian cells. In certain embodiments, such protein may be used, for example, as a therapeutic protein of the invention. For example, a protein which is capable of binding to an RGS protein of the invention (e.g. RGS2, RGS4 or RGSz) and inhibiting the activity of the RGS protein is useful as a protein therapeutic of the invention. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein, Such enzymes, and their cognate recognition sequences, include Factor Xa, thrombin and enterokinase. Typical fusion expression vectors include pGEX (Pharmacia Biotech Inc; Smith, D, B. and Johnson, K. S. (1988) Gene 67:31-40), pMAL (New England Biolabs, Beverly, Mass.) and pRITS (Pharmacia, Piscataway, N.J.) which fuse glutathione S transferase (GST), maltose E binding protein, or protein A, respectively, to the target recombinant protein.

Purified fusion proteins can be utilized in screening assays, (e.g., direct assays or competitive assays described in detail below), or to generate antibodies specific for RGS or Gα proteins.

Examples of suitable inducible non-fusion E. coli expression vectors include pTrc (Hmann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HSLE174(DE3) from a resident prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the polynucleotide sequence of the polynucleotide to be inserted into an expression vector so that the individual codons for each amino acid are those preferentially utilized in E. coli (Wade et al., (1992) Polynucleotides Res. 20:2111-2118). Such alteration of polynucleotide sequences of the invention can be carried out by standard DNA synthesis techniques.

In another embodiment, the expression vector is a yeast expression vector. Examples of vectors for expression in yeast S. cerevisiae include pYepSec1 (Baldari, et a/., (1987) Embo J. 6:229-234), pMFa (Kurjan and Herskowitz, (1982) Cell 30:933-943), pJRY88 (Schultz et al., 21987) Gene 54:113-123); pYES2 (InVitrogen Corporation. San Diego, Calif.), and picZ (Invitrogen Corp; San Diego, Calif.).

Alternatively, polynucleotides of the invention can be expressed in insect cells using baculovirus expression vectors. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a polynucleotide of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed.. Cold Spring Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HSLE174(DE3) from a resident prophage harboring a T7 gn1 gene under the transcriptional control of the lacUV 5 promoter.

In another embodiment, the recombinant mammalian expression vector is capable of directing expression of the polynucleotide preferentially in a particular cell type (e.g., tissue-specific regulatory elements are used to express the polynucleotide). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748).. neuron-specific promoters (e.g., the neurofilament promoter, Byrne and R.aaddle (1989) Proc. Nall. Acad Sci. USA 86:5473-5477), pancreas-specific promoters (Edlund et al. (1985) Science 230:912-916), and mammary gland-specific promoters (e.g., milk whey promoter, U.S. Pat. No. 4,873,316 and European Application Publication No. 264,166). Developmentally-regulated promoters are also encompassed, for example the marine hox promoters (Kessel and Grass (1990) Science 249:374-379) and the α-fetoprotein promoter (Campes and Tilghman (1989) Genes Dev. 3:537-546). In preferred embodiments of the invention, the promoter is a neuron-specific promotor.

The invention further provides a recombinant expression vector comprising a polynucleotide of the invention cloned into the expression vector in an antisense orientation. That is, the DNA molecule is operatively linked to a regulatory sequence in a manner which allows for expression (by transcription of the DNA molecule) of an RNA molecule which is antisense to mRNA corresponding to a RGS or Gα gene of the invention. Regulatory sequences operatively linked to a polynucleotide cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense polynucleotides are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1)1986.

Another aspect of the invention pertains to host cells into which a polynucleotide molecule of the invention is introduced, e.g., a gene encoding a protein listed in Table 1, or homolog thereof, within a recombinant expression vector or a polynucleotide molecule of the invention containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences; such progeny may not, in fact, be identical to the parent cell, but are still included within the scope of the term as used herein.

A host cell can be any prokaryotic or eukaryotic cell. For example, a RGS or Gα protein of the invention can be expressed in bacterial cells such as E. coli, insect cells, yeast or mammalian cells (such as Chinese hamster ovary cells (CHO) or COS cells). Other suitable host cells are known to those skilled in the art. In certain embodiments of the invention, the host cell is preferably a eukaryotic cell, most preferably a mammalian cell.

Vector DNA can be introduced into prokaryotic or eukaryotic cells via conventional transformation or transfection techniques. As used herein, the terms “transformation” and “transfection” are intended to refer to a variety of art-recognized techniques for introducing foreign polynucleotide (e.g., DNA) into a host cell, including calcium phosphate or calcium chloride co-precipitation, DAKD-dextran-mediated transfection, lipofection, or electoporation. Suitable methods for transforming or transferring host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y.,1989), and other laboratory manuals known in the art.

For stable transfection of mammalian cells, it is known that, depending upon the expression vector and transfection technique used, only a small fraction of cells may integrate the foreign DNA into their genome. In order to identify and select these integrants, a gene that encodes a selectable flag (e.g., resistance to antibiotics) is generally introduced into the host cells along with the gene of interest. Preferred selectable flags include those which confer resistance to drugs, such as G418, hygromycin and methotrexate. Polynucleotide encoding a selectable flag can be introduced into a host cell on the same vector as that encoding RGS or Gα protein of the invention or can be introduced on a separate vector. Cells stably transfected with the introduced polynucleotide can be identified by drug selection (e.g., cells that have incorporated the selectable flag gene will survive, while the other cells die).

A host cell of the invention, such as a prokaryotic or eukaryotic host cell in culture, can be used to produce (i.e., express) an RGS or Gα protein of the invention. Accordingly the invention further provides methods for producing proteins using the host cells of the invention. In one embodiment the method comprises culturing the host cell of invention (into which a recombinant expression vector encoding a marker protein has been introduced) in a suitable medium such that a RGS or Gα protein of the invention is produced. In another embodiment, the method further comprises isolating the protein from the medium or the host cell.

Detection Methods

Detection and measurement of the relative amount of a polynucleotide or polypeptide of the invention may be by any method known in the art (see, i.e., Sambrook, Fritsh and Maniatis, Molecular Cloning: A Laboratory Manual. 2^{nd,} ed., Cold Spring Harbor Laboratory, Cold Spring Harbor Laboratory Press, Cold Spring Harbor, N.Y. (1989), and Current Protocols in Molecular Biology, eds. Ausubel et al, John Wiley & Sons (1992)).

Typical methodologies for detection of a transcribed polynucleotide include RNA extraction from a cell or tissue sample, followed by hybridization of a labeled probe (i.e., a complementary polynucleotide molecule) specific for the target RNA to the extracted RNA and detection of the probe (ie. Northern blotting).

Typical methodologies for peptide detection include protein extraction from a cell or tissue sample, followed by binding of an antibody specific for the target protein to the protein sample, and detection of the antibody (such as Western blotting, or ELISA). Antibodies are generally detected by the use of a labeled secondary antibody. The label can be a radioisotope, a fluorescent compound, an enzyme, an enzyme co-factor, or ligand. Such methods are well understood in the art.

In certain embodiments, the genes (encoding an RGS or Gα protein) themselves (i.e., the DNA or cDNA) may serve as markers for a GPCR-related disorder. For example, an increase of polynucleotide corresponding to an RGS or Gα protein, such as by duplication of the gene, may also be correlated with a GPCR-related disorder since this increase may be associated with decreased GPCR signaling.

Detection of specific polynucleotide molecules may also be assessed by gel electrophoresis, column chromatography, or direct sequencing, or quantitative PCR (in the case of polynucleotide molecules) among many other techniques well known to those skilled in the art.

Detection of the presence or number of copies of ail or a part of a RGS or Gα gene of the invention may be performed using any method known in the art. Typically, it is convenient to assess the presence and/or quantity of a DNA or cDNA by Southern analysis, in which total DNA from a cell or tissue sample is extracted, hybridized with a labeled probe (i.e. a complementary DNA molecules), and the probe is detected. The label group can be a radioisotope, a fluorescent compound, an enzyme, or an enzyme co-factor. Other useful methods of DNA detection and/or quantification include direct sequencing, gel electrophoresis, column chromatography, and quantitative PCR, as is known by one skilled in the art.

In certain embodiments, the RGS or Gα proteins or polypeptides of the invention may serve as markers for a GPCR-related disorder. For example, an aberrent increase in the polypeptide corresponding to a RGS protein, may also be correlated with a GPCR-related disease.

Detection of specific polypeptide molecules may also be assessed by gel electrophoresis, column chromatography, western analysis or direct sequencing, among many other techniques well known to those skilled in the art.

A preferred agent for detecting an RGS or Gα protein is an antibody capable of binding to the protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect mRNA, protein, or genomic DNA in a biological sample in vitro as well as in vivo. For example, in vitro techniques for detection of mRNA include Northern hybridizations and in situ hybridizations. In vitro techniques for detection of protein include enzyme linked immunosorbent assays (ELISAs)., Western blots, immunoprecipitations and immunofiuorescence. In vitro techniques for detection of marker genomic DNA include Southern hybridizations. Furthermore, in vivo techniques for detection of proteins include introducing into a subject a labeled antibody. For example, the antibody can be labeled with a radioactive marker whose presence and location in a subject can be detected by standard imaging techniques.

The methods of the invention can also be used to detect genetic alterations in a RGS or Gα gene, thereby determining if a subject with the altered gene is at risk for damage characterized by aberrant regulation in marker protein activity or polynucleotide expression. In preferred embodiments, the methods include detecting, in a sample of cells from the subject, the presence or absence of a genetic alteration characterized by at least one alteration affecting the integrity of a gene encoding a RGS or Gα, or the aberrant expression of the gene. For example, such genetic alterations can be detected by ascertaining the existence of at least one of the following: 1) deletion of one or more nucleotides from the gene; 2) addition of one or more nucleotides to the gene; 3) substitution of one or more nucleotides of the gene; 4) a chromosomal rearrangement of the gene; 5) alteration in the level of a messenger RNA transcript of the gene; 6) aberrant modification of the gene, such as of the methylation pattern of the genomic DNA; 7) the presence of a non-wild type splicing pattern of a messenger RNA transcript of the gene; 8) non-wild type level of the encoded protein; 9) allelic loss of the gene; and 10) inappropriate post-translational modification of the encoded protein. As described herein, there are a large number of assays known in the art which can be used for detecting alterations in a gene such as an RGS or Gα gene of the invention.

In certain embodiments, detection of the alteration involves the use of a probe/primer in a polymerase chain reaction (PCR) (see, e.g., U.S. Pat. No. 4,683,995 and U.S. Pat. No. 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Mail. Acad. Sci. USA 91:360-364), the latter of which can be particularly useful for detecting point mutations in the marker-gene (see Abravaya et al. (1995) Polynucleotides Res. 23:675-682). This method can include the steps of collecting a sample of cells from a subject. isolating polynucleotide (e.g.. genomic. mRNA or both) from the cells of the sample, contacting the polynucleotide sample with one or more primers which specifically hybridize to a gene of interest under conditions such that hybridization and amplification of the gene of interest (if present) occurs, and detecting the presence or absence of an amplification product, or detecting the size of the amplification product and comparing the length to a control sample. It is understood that PCR and/or LCR may be desirable to use as a preliminary amplification step in conjunction with any of the techniques used for detecting mutations described herein.

Alternative amplification methods include: self sustained sequence replication (Guatelli, J C. et al., (1990) Proc. Natl. Acad. Sci. USA 87:1874-1878), transcriptional amplification system (Kwoh, D. Y. et al., (1989) Proc. Natl. Acad. Sci. USA 86:1173-1177), Q-Beta Replicase (Lizardi, P. M. et al. (1988) Bio-Technology 6:1197), or any other polynucleotide amplification method, followed by the detection of the amplified molecules using techniques well known to those of skill in the art. These detection schemes are especially useful for the detection of polynucleotide molecules if such molecules are present in very low numbers.

In an alternative embodiment, mutations in a gene such as on RGS or Gα of the invention from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in a gene of the invention can be identified by hybridizing a sample and control polynucleotides, e.g., DNA or RNA, to high density arrays containing hundreds or thousands of oligonucleotides probes (Cronin, M. T. et al. (1996) Human Mutation 7: 244-255; Kozal, M. J. et al. (1996) Nature Medicine 2: 753-759). For example, genetic mutations can be identified in two dimensional arrays containing light generated DNA probes as described in Cronin. M. T. et al. supra. Briefly; a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence a gene of the invention and detect mutations by comparing the sequence of the gene in a test sample with a corresponding wild-type (control) sequence. Examples of sequencing reactions include those based on techniques developed by Maxam and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Biotechniques 19:448), including sequencing by mass spectrometry (see, e.g., PCT International Publication No. WO 94/116101; Cohen et al. (1996) Adv. Chromatogr. 36:127-162; and Griffin et al. (1993) Appl. Biochem. Biotechnol. 38:147-159).

Other methods for detecting mutations in a gene of the invention include methods in which protection from cleavage agents is used to detect mismatched bases in RNA/RNA or RNA/DNA heteroduplexes (Myers et al. (1985) Science 230:1242). In general, the technique of “mismatch cleavage” starts by providing heteroduplexes by hybridizing (labeled) RNA or DNA containing the wild-type sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl Acad Sci USA 85:4397; Saleeba et al. (1992) Methods Enzymol. 517:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1652). According to an exemplary embodiment, a probe based on a RGS sequence, e.g., a wild-type RGS sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in genes of the invention. For example, single strand conformation polymorphism (SSCP) may be used to detect differences in electrophoretic mobility between mutant and wild type polynucleotides (Orita et al. (1989) Proc Natl. Acad. Sci. USA: 86:2766, see also Cotton (1993) Mutat. Res. 285:125-144; and Hayashi (1992) Genet. Anal. Tech Appl. 9:73-79). Single-stranded DNA fragments of sample and control polynucleotides will be denatured and allowed to renature. The secondary structure of single-stranded polynucleotides varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment the movement of mutant or wild-type fragments in polyacrylamide gels containing a gradient of denaturant is assayed using denaturing gradient gel electrophoresis (DGGF) (Myers et al. (1985) Nature 313: 495) When DGGE is used as the method of analysis; DNA will be modified to insure that it does not completely denature, for example by adding a GC clamp of approximately 40 by of high-melting GC-rich DNA by PCR. in a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl. Acad. Sci USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Polynucleotides Res. 17:2437-2448) or at the extreme 3′ end of one primer where, under appropriate conditions, mismatch can prevent, or reduce polymerase extension (Prossner (1993) Tibtech 11:238). In addition, it may be desirable to introduce a novel restriction site in the region of the mutation to create cleavage-based detection (Gasparini et al. (1992) Mol. Cell Probes 6:1). In certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

Screening

The invention also provides methods (also referred to herein as “screening assays”) for identifying modulators., i.e. candidate or test compounds or agents comprising therapeutic moieties (e.g., peptides, peptidomimetics, peptoids, polynucleotides, small molecules or other drugs) which (a) bind to an RGS, or (b) have an inhibitory effect on the activity of a marker or, more specifically, (c) have a modulatory effect on the interactions of the RGS with one or more of its natural substrates (e.g., Gαi or Gαq), or (d) have an inhibitory effect on the expression of the RGS. Such assays typically comprise a reaction between the RGS and one or more assay components. The other components may be either the test compound itself, or a combination of test compound and a binding partner of the RGS.

The test compounds of the present invention are generally either small molecules or bioactive agents. In one preferred embodiment, the test compound is a small molecule. In another preferred embodiment, the test compound is a bioactive agent. Bioactive agents include, but are not limited to, naturally-occurring or synthetic compounds or molecules (“biomolecules”) having bioactivity in mammals, as well as proteins, peptides, oligopeptides, polysaccharides, nucleotides and polynucleotides. Preferably, the bioactive agent is a protein, polynucleotide or biomolecule. One skilled in the art will appreciate that the nature of the test compound may vary depending on the nature of the protein encoded by the RGS of the invention. The test compounds of the present invention may be obtained from any available source, including systematic libraries of natural and/or synthetic compounds.

Methods and compositions for screening for protein inhibitors or activators are known in the art (see U.S. Pat. No. 4,980,281, U.S. Pat. No. 5,266,464, U.S. Pat. No. 5,688,635, and U.S. Pat. No. 5,877,007, which are incorporated herein by reference), and may be used in combination with the methods of the invention.

Screening for Inhibitors of GPCR-Related Disorders

The invention provides methods of screening test compounds for inhibitors of GPCR-related disorders, and to the pharmaceutical compositions comprising the test compounds capable of inhibition of an RGS molecule. One method of screening comprises obtaining samples from subjects diagnosed with or suspected of having a GPCR-related disorder, contacting each separate aliquot of the samples with one of a plurality of test compounds. and comparing expression of one or more RGS and Gα protein in each of the aliquots to determine whether any of the test compounds provides: a substantially decreased level of expression or activity of a RGS protein relative to samples with other test compounds or relative to an untreated sample or control sample. In addition, methods of screening may be devised by combining a test compound with a protein and thereby determining the effect of the test compound on the protein.

In addition, the invention is further directed to a method of screening for test compounds capable of inhibiting the binding of a RGS protein and a Gα protein, by combining the test compound, RGS protein, and Gα protein together and determining whether binding of the RGS protein and Gα protein occurs in the presence of the test compound. The test compounds may be either small molecules or bioactive agents. As discussed below, test compounds may be provided from a variety of libraries well known in the art.

In a specific embodiment the screening assay involves detection of a test compound's ability to inhibit the binding of a RGS protein to Gα protein. Such compounds may provide therapeutic agents of the invention useful for the treatment of GPCR-related disorders.

Inhibitors of RGS expression, activity or binding ability are useful as thereapeutic compositions of the invention. Such inhibitors may be formulated as pharmaceutical compositions, as described herein below. Such inhibitors may also be used in the methods of the invention, for example, to diagnose, treat, or prognose a GPCR-related disorder.

One embodiment of the invention provides a method of assessing the efficacy of a test compound for inhibiting a GPCR-related disorder in a subject. The method includes contacting a test cell with one of a plurality of test compounds in the presence of a GPCR agonist; detecting the expression of the reporter gene; and comparing the expression of the reporter gene in the test cell contacted by the test compound with the expression of the reporter gene in a test cell contacted by the agonist in the absence of the test compound, where a substantially increased level of expression of the reporter gene in the test cell contacted by the test compound and agonist, relative to the expression of the reporter gene in the test cell contacted by the agonist, is an indication that the test compound is efficacious for inhibiting the GPCR-related disorder in the subject. In this embodiment, the test cell includes a GPCR, an RGS protein, a corresponding Gα protein expressed at a level capable of attenuating GPCR-signaling by at least 50% as compared to a cell without the Gα protein expression level, and a reporter gene.

In another embodiment, the invention provides a method of screening test compounds for inhibitors of a GPCR-related disorder in a subject. The method includes the steps of obtaining a sample of cells from a subject; contacting an aliquot of the sample with one of a plurality of test compounds; detecting the expression levels RGS protein and Gα protein in each of the aliquots; and selecting one of the test compounds which substantially inhibits expression of a RGS protein expression in the aliquot containing that test compound, relative to other test compounds.

In another embodiment, the invention provides a method of screening test compounds for inhibitors of a GPCR-related disorder in a subject. The method includes the steps of obtaining a sample of cells from a subject; contacting an aliquot of the sample with one of a plurality of test compounds; detecting the activity of RGS and Gα protein in each of the aliquots; and selecting one of the test compounds which substantially inhibits activity of an RGS protein in the aliquot containing that test compound, relative to other test compounds.

In another embodiment, the invention provides a method of screening for a test compound capable of interfering with the binding of an RGS protein and a Gα. The method includes combining an RGS protein, a test compound, and a Gα; determining the binding of the RGS protein and the Gα; and correlating the ability of the test compound to interfere with binding, where a decrease in binding of the RGS protein and the Gα in the presence of the test compound as compared to the absence of the test compound indicates that the test compound is capable of inhibiting binding.

High-Throughput Screening Assays

The invention provides methods of conducting high-throughput screening for test compounds capable of inhibiting activity or expression of a RGS protein of the invention. In one embodiment, the method of high-throughput screening involves combining test compounds and a RGS protein in the presence of Gα protein and detecting the effect of the test compound on the RGS protein.

In one embodiment. the present invention provides a method of high-throughput screening for test compounds capable of inhibiting an RGS protein. The method includes: a) contacting a test cell with one of a plurality of test compounds in the presence of a GPCR agonist, wherein the test cell includes a GPCR, a RGS protein, a corresponding Gα protein expressed at a level capable of attenuating GPCR-signaling by at least 50% as compared to a cell without said Gα protein expression level, and a reporter gene; b) detecting the expression of the reporter gene in the test cell contacted by a test compound relative to other test compounds; and c) correlating the amount of expression level of the reporter gene with the ability of the test compound to inhibit RGS, where increased expression of the reporter gene indicates that the test compound is capable of inhibiting the RGS protein.

In another embodiment, the present invention provides a method of high-throughput screening for test compounds capable of inhibiting a GPCR-related disorder in a subject. The method includes the steps of: a) combining an RGS protein, Gα, and a test compound; b) detecting binding of the RGS protein and Gα in the presence of a test compound; and c) correlating the amount of inhibition of binding between RGS and Gα with the ability of the test compound to inhibit the GPCR-related disorder, where inhibition of binding of the RGS protein and Gα indicates that the test compound is capable of inhibiting the GPCR-related disorder.

Functional assays such as cytosensor microphysiometer, calcium flux assays such as FLIPR® (Molecular Devices Corp, Sunnyvale, Calif.), or the TUNEL assay may be employed to measure cellular activity, as discussed below.

A variety of high-throughput functional assays well-known in the art may be used in combination to screen and/or study the reactivity of different types of activating test compounds, but since the coupling system is often difficult to predict, a number of assays may need to be configured to detect a wide range of coupling mechanisms. A variety of fluorescence-based techniques are well-known in the art and are capable of high-throughput and ultra high-throughput screening for activity, including, but not limited, to BRET® or FRET® (both by Packard Instrument Co., Meriden, Conn.). A preferred high-throughput screening assay is provided by BIACORE® systems, which utilizes label-tree surface plasmon resonance technology to detect binding between a variety of bioactive agents, as described in further detail below. The ability to screen a large volume and a variety of test compounds with great sensitivity permits analysis of the potential RGS inhibitors and inhibitors of GPCR-related disorders. The BIACORE® system may also be manipulated to detect binding of test compounds with individual components such as an RGS.

Recent advancements have provided a number of methods to detect binding activity between bioactive agents. Common methods of high-throughput screening involve the use of fluorescence-based technology, including, but not limited, to BRET® or FRET® (both by Packard Instrument Co., Meriden, Conn.) which measure the detection signal provided by the proximity of bound fluorophores. By combining test compounds with the RGS proteins and/or the Gα proteins of the invention and determining the binding activity between such, diagnostic analysis can be performed. Generic assays using cytosensor microphysiometer may also be used to measure metabolic activation, while changes in calcium mobilization can be detected by using the fluorescence-based techniques such as FLIPR® (Molecular Devices Corp, Sunnyvale, Calif.). In addition, the presence of apoptotic cells may be determined by TUNEL assay, which utilizes flow cytometry to detect free 3 —OH termini resulting from cleavage of genomic DNA during apoptosis. As mentioned above, a variety of functional assays well-known in the art may be used in combination to screen and/or study the reactivity of different types of activating test compounds. Preferably, the high-throughput screening assay of the present invention utilizes label-free plasmon resonance technology as provided by BIACORE® systems (Biacore International AB, Uppsala, Sweden). Plasmon free resonance occurs when surface plasmon waves are excited at a metal/liquid interface. By reflecting directed light from the surface as a result of contact with a sample, the surface plasmon resonance causes a change in the refractive index at the surface layer. The refractive index change for a given change of mass concentration at the surface layer is similar for many bioactive agents (including proteins, peptides, lipids and polynucleotides), and since the BIACORE® sensor surface can be functionalized to bind a variety of these bioactive agents, detection of a wide selection of test compounds can thus be accomplished.

Therefore, in certain embodiments the invention provides for high-throughput screening of test compounds for the ability to inhibit activity of the RGS proteins listed in Table 1, by combining the test compounds and the protein in high-throughput assays such as BIACORE®, or in fluorescence based assays such as BRET®.

In a specific embodiment, the high-throughput screening assay detects the ability of a plurality of test compounds to bind to RGS protein. In another specific embodiment, the high-throughput screening assay detects the ability of a plurality of a test compound to inhibit a RGS binding partner (such as Gα protein) to bind to RGS protein. In yet another specific embodiment, the high-throughput screening assay detects the ability of a plurality of a test compounds to modulate signaling through GPCR.

Predictive Medicine

The present invention pertains to the field of predictive medicine in which diagnostic assays, prognostic assays, pharmacogenetics and monitoring clinical trials are used for prognostic (predictive) purposes to thereby treat an individual prophylactically. Accordingly, one aspect of the present invention relates to diagnostic assays for determining marker polynucleotide and/or polypeptide expression and/or activity, in the context of a biological sample (e.g., blood, serum, cerebral spinal fluid, cells, tissue) to thereby determine whether an individual is at risk for developing a GPCR-related disorder associated with decreased GPCR-signaling. The invention also provides for prognostic (or predictive) assays for determining whether an individual is at risk of developing a GPCR-related disorder associated with increased RGS or Gα protein or polynucleotide expression or activity.

For example, the number of copies of a RGS or Gα gene can be assayed in a biological sample. Such assays can be used for prognostic or predictive purposes to thereby phophylactically treat an individual prior to the onset of a GPCR-related disorder, characterized by, or-associated with, increased RGS protein, polynucleotide expression or activity.

Another aspect of the invention pertains to monitoring the influence of agents (e.g., drugs, compounds) on the expression or activity of marker in clinical trials.

Diagnostic Assays

An exemplary method for detecting the presence or absence of RGS or Gα protein or polynucleotide of the invention in a biological sample involves obtaining a biological sample from a test subject and contacting the biological sample with a compound or an agent capable of detecting the RGS or Gα protein or polynucleotide (e.g.; mRNA; genomic DNA) such that the presence of the protein or polynucleotide is detected in the biological sample. A preferred agent for detecting mRNA or genomic DNA corresponding to a polynucieotide of the invention is a labeled polynucleotide probe capable of hybridizing to a mRNA or genomic DNA of the invention. Suitable probes for use in the diagnostic assays of the invention are described herein. A preferred agent for detecting a marker protein of the invention is an antibody which specifically recognizes the protein.

The diagnostic assays may also be used to quantify the amount of expression or activity of a marker in a biological sample. Such quantification is useful, for example, to determine the progression or severity of a GPCR-related disorder. Such quantification is also useful, for example, to determine the severity of a GPCR-related disorder following treatment.

Determining Severity of a GPCR-Related Disorder

In the field of diagnostic assays, the invention also provides methods for determining the severity of a GPCR-related disorder by isolating a sample from a subject (e.g., a blood sample containing cells expressing GPCR), detecting the presence, quantity and/or activity of one or more RGS or Gα molecules of the invention in the sample relative to a second sample from a normal sample or control sample. In one embodiment, the levels of RGS protein in the two samples are compared, and a increase in the test sample compared to the normal sample indicates a GPCR-related disorder. In other embodiments the modulation of 2, 3, 4 or more RGS proteins indicate a severe GPCR-related disorder.

In one embodiment, the present invention provides a method of determining the severity of a GPCR-related disorder in a subject by comparing; a) a level of expression of RGS protein in a sample from the subject; and b) a normal level of expression of RGS protein in a control sample, where an abnormal level of expression of RGS protein in the sample from the subject relative to the normal levels is an indication that the subject is suffering from a severe GPCR-related disorder.

In another embodiment, the present invention provides a method of assessing the efficacy of a therapy for inhibiting a GPCR-related disorder in a subject by comparing; a) expression of a RGS protein in a first sample obtained from the subject prior to providing at least a portion of the therapy to the subject, and b) expression of a RGS protein in a second sample following provision of the portion of the therapy, where a substantially modulated level of expression of the RGS protein in the second sample, relative to the first sample, is an indication that the therapy is efficacious for inhibiting the GPCR-related disorder in the subject.

In another embodiment, the present invention provides a method for diganosisng a GPCR-related disorder by; a) obtaining a sample from a subject comprising cells; b) measuring the expression of RGS and Gα in the sample; c) correlating the amount of RGS and Gα with the presence of a GPCR-related disorder, where the substantially increased levels of RGS and Gα as compared to a control sample are indicative of the presence of GPCR-related disorder.

In one embodiment, the biological sample contains protein molecules from the test subject. Alternatively, the biological sample can contain mRNA molecules from the test subject or genomic DNA molecules from the test subject. A preferred biological sample is white blood cells isolated by conventional means from a subject.

In another embodiment, the methods further involve obtaining a control biological sample from a subject, contacting the control sample with a compound or agent capable of detecting an RGS or Gα protein, mRNA, or genomic DNA, such that the presence of RGS or Gα protein, mRNA or genomic DNA is detected in the biological sample, and comparing the presence of the same protein, mRNA or genomic DNA in the control sample.

Prognostic Assays

The diagnostic methods described herein can furthermore be utilized to identify subjects having, or at risk of developing, a GPCR-related disorder associated with decreased GPRC-signaling. In one embodiment of the present invention, as related to a GPCR-related disorder, increased expression or activity of RGS protein markers is typically correlated with a GPCR-related disorder.

The assays described herein, such as the preceding or following assays, can be utilized to identify a subject having a GPCR-related disorder associated with an increased level of RGS activity or expression. Alternatively, the prognostic assays can be utilized to identify a subject at risk for developing a GPCR-related associated with increasedtlevels of RGS protein activity or polynucleotide expression. Thus, the present invention provides a method for identifying GPCR-related disorders associated with increased RGS expression or activity in which a test sample is obtained from a subject and an RGS protein or polynucleotide (e.g., mRNA or genomic DNA) is detected, wherein the presence of increased RGS protein or polynucleotide is diagnostic or prognostic for a subject having or at risk of developing a GPCR-related disorder.

Furthermore, the prognostic assays described herein can be used to determine whether a subject can be administered an agent (e.g., peptidomimetic, protein, peptide, polynucleotide, small molecule, or other drug candidate) to treat or prevent a GPCR-related disorder. For example, such methods can be used to determine whether a subject can be effectively treated with an agent to inhibit a GPCR-related disorder. Thus, the present invention provides methods for determining whether a subject can be effectively treated with an agent for a disorder associated with decreased GPCR-signaling in which a test sample is obtained and RGS and Gα protein or polynucleotide expression or activity is detected (e.g., wherein the abundance of protein or polynucleotide expression or activity is diagnostic for a subject that can be administered the agent to treat injury associated with decreased GPCR-signaling).

One embodiment of the invention provides a method of assessing the efficacy of a test compound for inhibiting a GPCR-related disorder in a subject by comparing; a) expression of a RGS protein in the presence of Gα in a first cell sample, where the first cell sample is exposed to the test compound, and b) expression of a RGS protein in the presence of Gα in a second cell sample, where the second cell sample is not exposed to the test compound, and where a substantially decreased level of expression of the RGS protein in the first sample, relative to the second sample, is an indication that the test compound is efficacious for inhibiting the GPCR-related disorder in the subject.

In relation to the field of GPCR-related disorders, prognostic assays can be devised to determine whether a subject undergoing treatment for such disorder has a poor outlook for long term survival or disease progression. In a preferred embodiment, prognosis can be determined shortly after diagnosis, i.e. within a few days. By establishing expression profiles of different stages of the GPCR-related disorder. from onset to acute disease. an expression pattern may emerge to correlate a particular expression profile to increased likelihood of a poor prognosis. The prognosis may then be used to devise a more aggressive treatment program to avert a chronic GPCR-related disorder and enhance the likelihood of long-term survival and well being.

The methods described herein may be performed, for example, by utilizing prepackaged diagnostic kits comprising at least one probe polynucleotide or antibody reagent described herein, which may be conveniently used, e.g., in clinical settings to diagnose subjects exhibiting symptoms or family history of a disease or illness involving a RGS or Gα gene. In a specific embodiment of the invention, a mutation is detected in a RGS polynucleotide or RGS polypeptide. In a further specific embodiment, such RGS mutation is correlated with the prognosis or susceptibility of a subject to a GPCR-related disorder such as, for example, schizophrenia, bipolar disorder, anxiety, depression, cariachypertrophy, hypertension, thrombosis, arrhythmia, inflammation, compromised immune responses and the like.

Furthermore, any cell type or tissue in which a RGS or Gα is expressed may be utilized in the prognostic or diagnostic assays described herein.

Monitoring of Effects During Clinical Trials

Monitoring the influence of agents (e.g., drugs, small molecules, proteins, nucleotides) on the expression or activity of a RGS or Gα protein (e.g., the modulation of RGS protein involved in a GPCR-related disorder) can be applied not only in basic drug screening, but also in clinical trials. For example, the effectiveness of an agent determined by a screening assay, as described herein, to decrease RGS gene expression, protein levels, or downregulate activity, can be monitored in clinical trials. In such clinical trials, the expression or activity of a RGS gene, and preferably, other genes that have been implicated in, for example, RGS-associated damage (e.g., resulting from a GPCR-related disorder) can be used as a “read out” of the phenotype of a particular cell.

For example, and not by way of limitation, genes that are modulated in cells by treatment with an RGS inhibitor which modulates RGS activity (e.g., identified in a screening assay as described herein) can be identified. Thus, to study the effect of RGS inhibitors on GPCR-signaling, cells can be isolated and analyzed for the levels of expression of RGS and other genes implicated in the GPCR-signaling pathway The levels of gene expression (e.g., a gene expression pattern) can be quantified by Northern blot analysis or RT-PCR, as described herein, or alternatively, by measuring the amount of protein produced, by one of the methods as described herein, or by measuring the levels of activity of marker or other genes. In this way, the gene expression pattern of the GPCR signaling pathway can serve as a read-out, indicative of the physiological response of the cells to the agent. Accordingly, this response state may be determined before, and at various points, during treatment of the individual with the agent.

In a preferred embodiment, the present invention provides a method for monitoring the effectiveness of treatment of a subject with an agent (e.g., an agonist, antagonist, peptidomimetic, protein, peptide, polynucleotide, small molecule, or other drug candidate identified by the screening assays described herein) including the steps of: (i) obtaining a pre-administration sample from a subject prior to administration of the agent; (ii) detecting the level of expression of RGS and Gα proteins, mRNAs, or genomic DNAs in the pre-administration sample; (iii) obtaining one or more post-administration samples from the subject; (iv) detecting the level of expression or activity of the RGS and Gα proteins, mRNAs, or genomic DNAs in the post-administration samples; (v) comparing the level of expression or activity of the proteins, mRNAs, or genomic DNAs in the pre-administration sample with the marker proteins, mRNAs, or genomic DNAs in the post administration sample or samples; and (vi) altering the administration of the agent to the subject accordingly. For example, increased administration of the agent may be desirable to decrease expression or activity of an RGS. According to such an embodiment, RGS expression or activity may be used as an indicator of the effectiveness of an agent, even in the absence of an observable phenotypic response.

Prophylactic Methods

In one aspect, the invention provides a method for preventing in a subject, a GPCR-related disorder associated with increased RGS expression or activity, by administering to the subject an agent which inhibits an RGS protein expression or activity.

Subjects at risk for a disease which is caused or contributed to by aberrant RGS expression or activity can be identified by.. for example. any or a combination of. diagnostic or prognostic assays as described herein.

Administration of a prophylactic agent can occur prior to the manifestation of symptoms characteristic of the GPCR-related disorder, such that the GPCR-related disorder is prevented or, alternatively, delayed in its progression. The appropriate agent can be determined based on screening assays described herein.

In another aspect, the invention provides a method for preventing in a subject a GPCR-related disorder by administering to the subject an agent which inhibits RGS protein expression or activity. One of skill in the art will appreciate that, with respect to embodiments for treating or preventing GPCR-related disorders, therapeutic or prophylactic methods generally seek to inhibit RGS protein expression or activity. As such, antagonists of RGS protein may be administered to effectuate such results. Appropriate agents for such use may be determined based on screening assays described herein.

Therapeutic Methods

Another aspect of the invention pertains to methods of inhibiting RGS protein expression or activity for therapeutic purposes. Accordingly, in an exemplary embodiment, the inhibitory method of the invention involves contacting a cell with an agent that modulates one or more of the activities of a RGS protein activity associated with the cell. An agent that modulates RGS protein activity can be an agent as described herein, such as a polynucleotide or a protein, a naturally-occurring target molecule of the protein (e.g., a RGS protein substrate), an antibody, an inhibitor, a peptidomimetic of a RGS protein antagonist, or other small molecule.

In one embodiment, the agent inhibits one or more RGS protein activities. Examples of such inhibitory agents include antisense RGS nucleic acid molecules, anti-RGS protein antibodies, and RGS protein inhibitors. In a specific embodiment, an inhibitor of agent is an anti-sense RGS polynucleotide, or RGS ribozyme.

In another embodiment of the invention, the RGS is abnormally increased in activity or expression levels in a subject diagnosed with, or suspected of having, an RGS-related disorder or a decreased expression of normal levels of Gα is desired. In this embodiment, treatment of such a subject may comprise administering an inhibitor of RGS wherein such inhibitor provides decreased activity or expression of Gα.

These modulatory methods can be performed in vitro (e.g., by culturing the cell with the agent) or, alternatively, in vivo (e.g., by administering the agent to a subject). As such, the present invention provides methods of treating an individual diagnosed with, or at risk for, a GPCR-related disorder characterized by aberrant expression or activity of one or more RGS and Gα proteins or polynucleotide molecules. In one embodiment, the method involves administering an agent (e.g., an agent identified by a screening assay described herein), or combination of agents that inhibits RGS protein expression or activity

The invention further provides methods of modulating a level of expression of a RGS protein of the invention, comprising administration to a subject having a GPCR-related disorder a variety of compositions, including antisense oligonucleotides or ribozyme. The composition may be provided in a vector comprising a polynucleotide encoding the oligonucleotide or ribozyme. Alternatively, the expression levels of the markers of the invention may be modulated by providing an antibody, a plurality of antibodies or an antibody conjugated to a therapeutic moiety. Treatment with the antibody may further be localized to the tissue comprising the GPCR-related disorder.

One embodiment of the invention provides a method of treating a subject diagnosed with a GPCR-related disorder by administering a composition including: a) an RGS inhibitor which specifically binds to an RGS protein; b) a Gα inhibitor which specifically binds to a Gα protein; and c) a pharmaceutically acceptable carrier.

In another embodiment, the invention provides a method of treating a subject diagnosed with a GPCR-related disorder. The method includes administering a composition including: a) an antisense oligonucleotide complementary to an RGS polynucleotide; b) an antisense oligonucleotide complementary to a Gα polynucleotide; and c) a pharmaceutically acceptable carrier.

In another embodiment, the invention provides a method of treating a subject diagnosed with a GPCR-related disorder by administering a composition including: a) a ribozyme which is capable of binding an RGS polynucleotide; b) a ribozyme which is capable of binding a Gα polynucleotide; and c) a pharmaceutically acceptable carrier.

Determining Efficacy of a Test Compound or Therapy

The invention also provides methods of assessing the efficacy of a test compound or therapy for inhibiting a GPCR-related disorder in a subject. These methods involve isolating samples from a subject suffering from a GPCR-related disorder, who is undergoing treatment or therapy, and detecting the presence, quantity, and/or activity of one or more markers of the invention in the first sample relative to a second sample. Where a test compound is administered, the first and second samples are preferably sub-portions of a single sample taken from the subject, wherein the first portion is exposed to the test compound and the second portion is not. In one aspect of this embodiment, the RGS is expressed at a substantially increased level in the first sample, relative to the second. Most preferably, the level of expression in the first sample approximates (i.e., is less than the standard deviation for normal samples) the level of expression in a third control sample, taken from a control sample of normal tissue. In certain embodiments, the normal sample is derived from a tissue substantially free of a GPCR-related disorder.

Where the efficacy of a therapy is being assessed, the first sample obtained from the subject is preferably obtained prior to provision of at least a portion of the therapy, whereas the second sample is obtained following provision of the portion of the therapy. The levels of the RGS in the samples are compared, preferably against a third control sample as well, and correlated with the presence, risk of presence, or severity of the GPCR-related disorder. Most preferably, the level of RGS in the second sample approximates the level of expression of a third control sample. In the present invention, a substantially decreased level of expression of a RGS indicates that the therapy is efficacious for treating the GPCR-related disorder associated with inhibited signaling.

Pharmacogenomics

The protein and polynucleotide molecules of the present invention, as well as inhibitors or agents that have an inhibitory effect on a RGS protein, as identified by a screening assay described herein, can be administered to individuals to treat (prophylactically or therapeutically) GPCR-related disorders.

In conjunction with such treatment (prophylactic or therapeutic) pharmacogenomics may be considered. “Pharmacogenomics,” as used herein, includes the application of genomics technologies, such as gene sequencing, statistical genetics, and gene expression analysis to drugs in clinical development and on the market. More specifically, the term refers to the study of how a subject's genes determine his or her response to a drug (e.g., a subject's “drug response phenotype”, or “drug response genotype”). Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer an agent as well as tailoring the dosage and/or therapeutic regimen of treatment.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11) :983-985 and Linden, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related sites (e.g., a “bi-allelic” gene marker map which consists of 60,000-100,000 polymorphic or variable sites on the human genome, each of which has two variants). Such a high-resolution genetic map can be compared to a map of the genome of each of a statistically substantial number of subjects taking part in a Phase II/III drug trial to identify genes associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drug target is known (e.g., a marker protein of the present invention), all common variants of that gene can be fairly easily identified in the population and it can be determined if having one version of the gene versus another is associated with a particular drug response.

Alternatively, a method termed the “gene expression profiling” can be utilized to identify genes that predict drug response. For example, the gene expression of an animal dosed with a drug (e.g., an RGS molecule of the present invention) can give an indication whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with a RGS inhibitor, such as one of the exemplary screening assays described herein.

Pharmaceutical Compositions

The invention is further directed to pharmaceutical compositions, which may be formulated as described herein. These compositions may include an RGS inhibitor, an antibody which specifically binds to a marker protein of the invention and/or an antisense polynucleotide molecule which is complementary to a RGS or Gα polynucleotide of the invention and can be formulated as described herein.

As used herein the language “pharmaceutically acceptable carrier” is intended to include any and all solvents, solubilizers, fillers, stabilizers, binders, absorbents, bases, buffering agents, lubricants, controlled release vehicles, diluents, emulsifying agents, humectants, lubricants, dispersion media, coatings, antibacterial or antifungal agents, isotonic and absorption delaying agents, and the like, compatible with pharmaceutical administration. The use of such media and agents for pharmaceutically active substances is well-known in the art. See e.g,. A. H. Kibbe, Handbook of Pharmaceutical Excipients, 3rd ed. Pharmaceutical Press, London, UK (2000). Except insofar as any conventional media or agent is incompatible with the active compound, use thereof in the compositions is contemplated. Supplementary agents can also be incorporated into the compositions.

The invention includes methods for preparing pharmaceutical compositions for modulating the expression or activity of a polypeptide or polynucleotide corresponding to a RGS or Gα of the invention. Such methods comprise formulating a pharmaceutically acceptable carrier with an agent which modulates expression or activity of a polypeptide or polynucleotide corresponding to a molecule of the invention. Such compositions can further include additional active agents. Thus, the invention further includes methods for preparing a pharmaceutical composition by formulating a pharmaceutically acceptable carrier with an agent which modulates expression or activity of a polypeptide or polynucleotide corresponding to a RGS of the invention and one or more additional bioactive agents.

One embodiment of the invention provides a composition capable of inhibiting a GPCR-related disorder in a subject, where the composition includes a therapeutically effective amount of an RGS inhibitor which specifically binds to an RGS protein; a Gα inhibitor which specifically binds to a Gα protein; and a pharmaceutically acceptable carrier.

In another embodiment, the invention provides a composition capable of inhibiting a GPCR-related disorder where the composition includes a therapeutically effective amount of an antisense oligonucleotide complementary to an RGS polynucleotide; an antisense oligonucleotide complementary to a Gα polynucleotide; and a pharmaceutically acceptable carrier.

In another embodiment, the invention provides a composition capable of inhibiting a GPCR-related disorder where the composition includes a therapeutically effective amount of a ribozyme which is capable of binding an RGS polynucleotide; a ribozyme which is capable of binding a Gα polynucleotide; and a pharmaceutically acceptable carrier.

A pharmaceutical composition of the invention is formulated to be compatible with its intended route of administration. Examples of routes of administration include parenteral (e.g., intravenous, intradermal, subcutaneous), oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine; propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the injectable composition should be sterile and should be fluid to the extent that easy syringability exists. It must be stable under the conditions of manufacture and storage and must be preserved against the contaminating action of microorganisms such as bacteria and fungi. The earner can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a fragment of a marker protein or an anti-marker protein antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Stertes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the bioactive compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the therapeutic moieties, which may contain a bioactive compound, are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from e.g. Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein includes physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The polynucleotide molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack or dispenser together with instructions for administration.

Kits

The invention also encompasses kits for detecting the presence of RGS or Gα proteins or polynucleotides in a biological sample. For example, the kit can comprise a labeled compound or agent capable of detecting the protein or mRNA in a biological sample; means for determining the amount of RGS or Gα in the sample; and means for comparing the amount in the sample with a control or standard. The compound or agent can be packaged in a suitable container. The kit can further comprise instructions for using the kit to detect marker protein or polynucleotide.

The invention also provides kits for determining the prognosis for long term survival in a subject having a GPCR-related disorder, the kit comprising reagents for assessing expression of the RGS and Gα molecules of the invention. Preferably, the reagents may be an antibody or fragment thereof, wherein the antibody or fragment thereof specifically binds with an RGS or Gα protein, respectively. For example, antibodies of interest may be commercially available, or may be prepared by methods known in the art. Optionally, the kits may comprise a polynucleotide probe wherein the probe specifically binds with a transcribed polynucleotide corresponding to a RGS or Gα polynucleotide.

The invention further provides kits for assessing the suitability of each of a plurality of compounds for inhibiting a GPCR-related disorder in a subject.

One embodiment of the present invention provides a kit for determining the long term prognosis in a subject having a GPCR-related disorder. The kit includes a first polynucleotide probe, wherein the probe specifically binds to a transcribed RGS polynucleotide, and a second polynucleotide probe, wherein the probe specifically binds to a transcribed Gα polynucleotide.

In another embodiment, the present invention provides a kit for determining the long term prognosis in a subject having a GPCR-related disorder where the kit includes a first antibody, wherein the first antibody specifically binds to a RGS polypeptide, and a second antibody, wherein the second antibody specifically binds to a corresponding Gα polypeptide.

In another embodiment, the present invention provides a kit for assessing the suitability of each of a plurality of compounds for inhibiting a GPCR-related disorder in a subject. The kit includes: a) a plurality of test cells, where each test cell comprises a GPCR, a RGS protein, a corresponding Gα protein expressed at a level capable of attenuating GPCR-signaling by at least 50% as compared to a cell without said Gα protein expression level, and a reporter gene, and b) an agonist for the GPCR.

Modifications to the above-described compositions and methods of the invention, according to standard techniques, will be readily apparent to one skilled in the art and are meant to be encompassed by the invention.

This invention is further illustrated by the following examples which should not be construed as limiting. The contents of all references, patents and published patent applications cited throughout this application, as well as the Figures and Tables are incorporated herein by reference.

### EXAMPLES

There is a need in the art for a drug screening assay for cells expressing GPCR, and particularly cells expressing Gαi. To address this need, an assay was developed that allows identification of potential drug candidates based on an interaction between an RGS protein and a Gα protein in cells expressing GPCRs. The interaction is quantified by comparing the expression of a reporter gene in a test cell contacted with a test compound with the expression of the reporter gene in a test cell contacted by an agonist of the GPCR.

As set forth below, results indicated that introduction of an RGS of the invention into the cell led to an inhibition of GPCR signaling by approximately 30-40% as compared to signaling without the RGS. Surprisingly, co-transfection of the RGS with a corresponding Gα protein lead to an inhibition of GPCR signaling by approximately 80-90%, as compared to signaling without the RGS or Gα molecules. Accordingly, Gαi or Gαq molecules in the presence of a corresponding RGS are capable of attenuating GPCR-signaling.

### Example 1

### Reagents

Pertussis toxin, quinpirole. PD098059 and wortmannin were purchased from Sigma (St. Louis, Mo.). Tissue culture reagents were purchased from Life Technologies, Inc (Gaithersburg, Md.). The luciferase/β-galatosidase reporter gene assay system was purchased from Tropix (Bedford, Mass.). Anti-phospho p44/42 polyclonal antibodies and anti-HRP-conjugated rabbit antibodies were purchased from Cell Signaling Technology (New England Biolabs, Bedford, Mass.). Anti-p42 polyclonal and anti-myc monoclonal antibodies were purchased from Santa Cruz Biotechnology, Inc.(Santa Cruz, Calif.). Anti-phospho-Akt polyclonal and anti-Akt monoclonal antibodies were purchased from Transduction Laboratories (San Diego, Calif.). Anti-HRP-conjugated mouse antibodies were purchased from Amersham Pharmacia Biotech (Piscataway, N.J.).

### Example 2

### DNA Constructs

The N-terminal myc-tagged and untagged human RGS2, RGS4, RGSz1, and Cdc42N17 were cloned into the eukaryotic expression vector pCR3.1 (InVitrogen, Carlsbad, Calif.), according to techniques known to those of ordinary skill in the art. Gαi1, Gαq/i chimera, and βARKct were cloned into the expression vector pcDNA3.1 (InVitrogen, Carlsbad, Calif.) according to techniques known to those of ordinary skill in the art. The respective N-terminal primers for myc-tagged RGS2, RGS4, and RGSz1 were:

| | |
|---|---|
| 5′-gccaccatggaacagaagctgatctccgaagaggacctcaacggcatgcaaagtgctatgttcttggctg-3′; | (SEQ ID NO:1) |
| 5′-ccaccatggaacagaagctgatctccgaagaggacctcaacggcatgtgcaaagggcttgcaggtc-3′; and | (SEQ ID NO:2) |
| 5′-ccaccatggaacagaagctgatctccgaagaggacctcaacggcatgggatcagagcggatggagatg-3′. | (SEQ ID NO:3) |

All expression constructs contained the Kozac (GCCACC) sequence before the ATG start codon to facilitate expression. Site-directed mutagenesis was carried out using the Quick-Change mutagenesis kit (Stratagene, La Jolla, Calif.). All constructs were verified by DNA sequencing of the entire protein coding region. Expression constructs of RhoN19, RacN17, and C3 exoenzyme were kindly provided by R. Herrara (University of Michigan). The reporter pCMV-βGal was kindly provided by Y. Dai (Columbia University) and the pSRE-luciferase reporter was purchased from Stratagene (La Jolla, Calif.).

### Example 3

### Cell Culture, Transfection and Luciferase Assays

CHO cells stably expressing D2R were grown and maintained in Dulbecco's Modified Eagle's medium supplemented with 10% fetal calf serum, non-essential amino acids, penicllin/streptomycin, 5 μg/ml mycophenolic acid, 0.25 mg/ml xanthine, and HT supplement. Cells were split into 6-well plates the day before transfection and grown to 40-60% confluence on the day of transfection. Transient transfection was performed using LipofectAMINE Plus™ reagent (Gibco Life Technologies, Inc., Gaithersburg, Md.) and carried out according to the manufacturer's instructions. Briefly, 5 μg of total DNA was used per plate and transfection was carried out in Optio-MEM™ medium with glutamine (Gibco Life Technologies, Inc., Gaithersburg, Md.). Three hours after transfection, an equal volume of growth medium (containing 10% fetal calf serum) was added to the transfection and cells were allowed to recover for 3-4 hours before being subjected to serum-free medium for 16 hours. The medium was then replaced with serum-free medium containing varying concentrations of quinpirole. After a 5 hours incubation, cell extracts were prepared and luciferase and β-galactosidase activities were measured using the dual reporter gene assay kit according to the manufacturer's instructions (Tropix, Bedford, Mass.).

### Example 4

### Western Blot Analysis

Cell lysates were prepared by incubating cells for 5 minutes on ice with a lysis buffer containing 1.50 mM NaCl, 50 mM Tris, pH 7.5, 5 mM EDTA, 1% Triton, and a mixture of protease inhibitors. Cells were then scraped off plates and sonicated. The detergent-insoluble material was removed by microcentrifugation for 10 minutes at 4° C. An equal amount of protein was run on SDS gels (Novex, Carlsbad, Calif.) and transferred to nitrocellulose (Bio-Rad, Hercules, Calif.). Membranes were blocked with 5% milk in TBS for 1 hour and incubated overnight in TBS containing 1% milk and an appropriate dilution of primary antibodies. The membrane was washed, incubated for 1 hour in TBS containing appropriate HRP-conjugated secondary antibodies, washed again, and developed with the ECL reagent (Amersham Pharmacia Biotech, Piscataway, N.J.).

### Example 5

### Activation of D2R Evokes the c-fos SRE Response Mediated by Gαγ Subunits

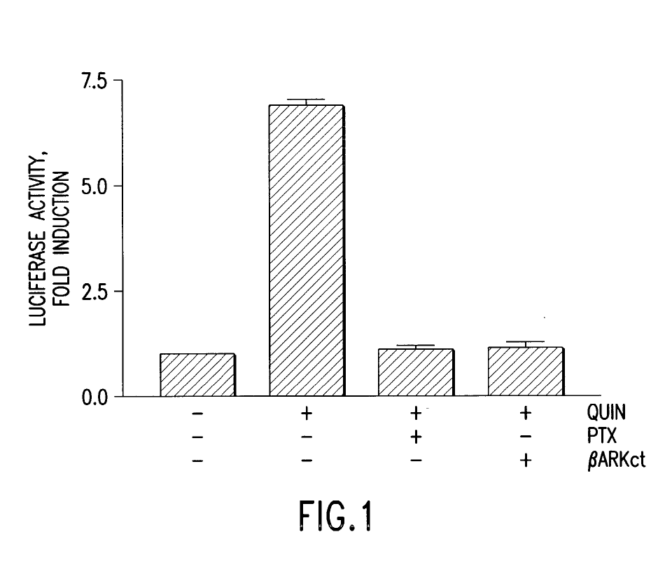

Activation of Gq-coupled and G_{12/13}-coupled receptors resulted in an activation of the c-fos SRE reporter in fibroblasts. Because activation is recapitulated by expressing constitutively active Gαq and Gα_{12/13} (See, Fromm et al., Proc. Natl. Acad. Sci. (1997) 94: 10098-10103; Mao et al., J. Biol. Chem. (1998) 273: 27118-27123), the finding established a role of Gαq and Gα_{12/13} in signaling to the SRE. While over-expression of Gβγ also results in the SRE activation, albeit with a lower magnitude (See, Fromm et al., Proc. Natl. Acad. Sci. (1997) 94: 10098-10103; Mao et al., J. Biol. Chem. (1998) 273: 27118-27123), it is controversial whether activation of a Gi-coupled receptor could induce the same transcriptional response (See, Mao et al., J. Biol. Chem. (1998) 273: 27118-27123; Sun et al., J. Biochem. (1999) 125: 515-521). To examine whether activation of D2R, a Gi-coupled receptor, stably expressed in CHO cells was able to initiate signaling events leading to the SRE activation, an SRE-luciferase reporter gene was transiently expressed. The luciferase activity was assayed following stimulation of cells with the D2R specific agonist, quinpirole. An approximately 7-fold induction of the luciferase activity was observed upon 10 μM of quinpirole treatment (FIG. 1). Pre-treatment of cells overnight with 10 ng/ml pertussis toxin (PTX) completely abolished the quinpirole-stimulated SRE activation (FIG. 1), confirming a Gi/o-mediated event. Transient expression of the β-adrenergic receptor kinase C-terminus (βARKct), which sequesters Gβγ from signaling to downstream effectors (See, Crespo et al., J. Biol. Chem. (1995) 270: 25259-25265) completely abrogated the SRE activation as well (FIG. 1). Thus, D2R-mediated SRE activation was initiated by Gβγ subunits thereby suggesting that activated Gαi does not signal to the SRE (See, Fromm et al., Proc. Natl. Acad. Sci. (1997) 94: 10098-10103; Mao et al., J. Biol. Chem. (1998) 273: 27118-27123).

### Example 6

### Expression of RGS Proteins Suppresses Quinpriole-Stimulated SRE Activation

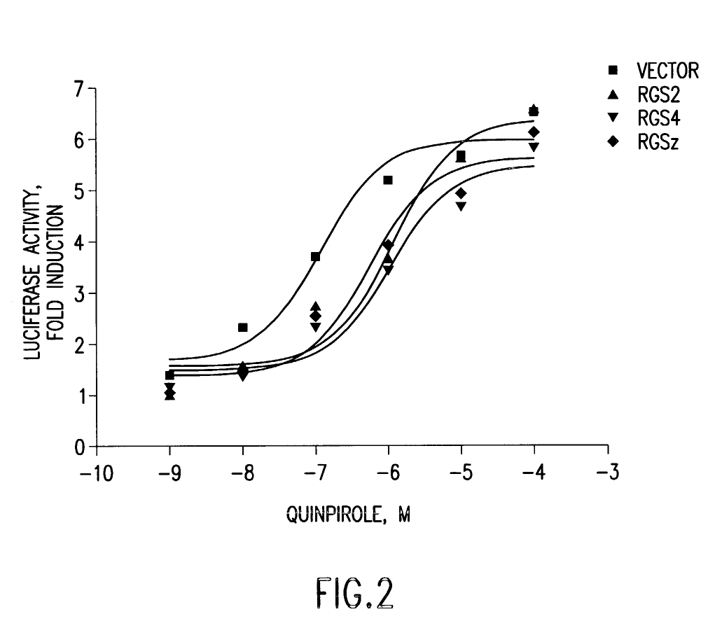

The proteins RGS2, RGS4, and RGSz1 were chosen to study the potential role of RGS proteins in quinpirole-induced SRE activation. These RGS proteins are composed primarily of the RGS domain and displayed distinct GAP profiles in vitro. RGS2 is a selective GAP for Gαq (See, Heximer et al., (1997) Proc. Natl. Acad. Sci. 94: 14389-14393), whereas RGS4 is a potent GAP for both Gαq and Gαi (See, Berman et al., (1996) Cell 86: 445-452; Hepler et al., (1997) Proc. Natl. Acad. Sci. 94: 428-432). RGSz1 is highly selective for Gαz, a member of Gαi family (Glick et al., (1997) J. Biol. Chem. 273: 26008-26013; Wang et al., (1997) J. Biol. Chem. 273: 26014-26025). As shown in FIG. 2, quinpirole stimulation of cells transiently transfected with control plasmids produced dose-dependent SRE activation. Transient transfection of the respective RGS proteins resulted in a similar degree of rightward shift of the dose-response curve. Western blot analysis of lysates from cells transfected with myc-tagged RGS2, RGS4, and RGSz1 demonstrated equivalent expression among the three RGS proteins. Thus, despite the differential Gαi GAP activity in vitro, the three RGS proteins equally attenuated D2R-initiated SRE activation in CHO cells.

### Example 7

### Expression of Gαi1 or Gαq/i Chimera Differentially Potentiates the Inhibition of RGS Proteins on Quinpirole-Induced SRE Activation

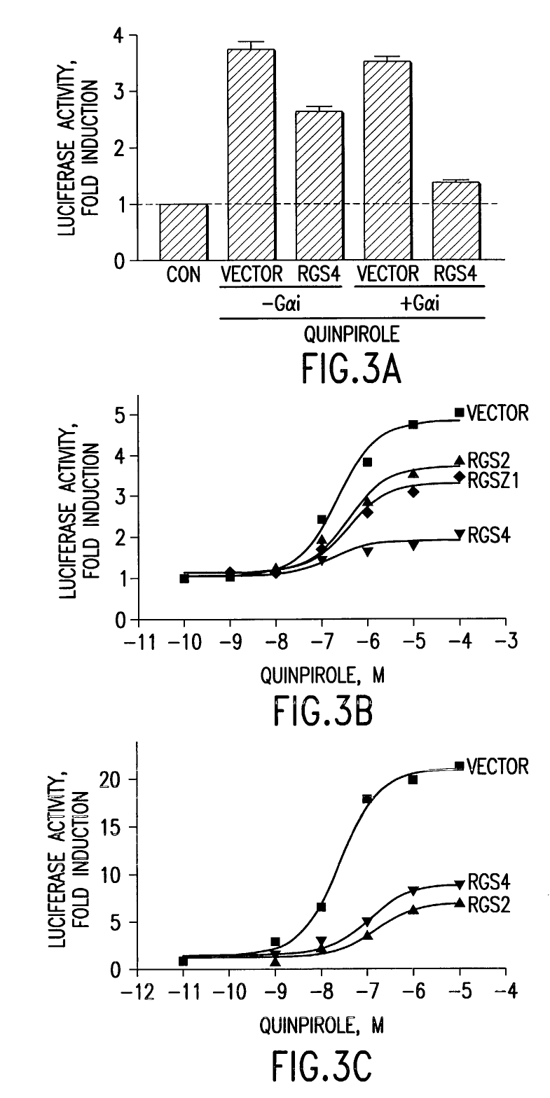

To test whether the available amount of Gα proteins would influence RGS activity in vivo, CHO-D2R cells were co-transfected with Gαi1 and RGS4. SRE activation was analyzed after stimulation with 100 nM of quinpirole. When Gαi1 by itself was overexpressed alone in the cell, a slightly lower magnitude of quinpirole-stimulated SRE activation was consistently observed as compared to cells expressing vector plasmids alone (FIG. 3A). The difference was more pronounced as higher concentrations of quinpirole were applied. Nevertheless, co-expression of RGS4 with Gαi1 resulted in approximately 85% reduction in quinpirole-stimulated SRE activation as compared to approximately 40% reduction observed with cells without Gαi1 over-transfection (FIG. 3A). To examine whether the Gαi1 potentiation was selective for different RGS proteins, CHO-D2R cells were transfected with Gαi1 and the three respective RGS proteins. While co-transfection of RGS4 with Gαi1 produced greater than approximately 80% inhibition of SRE activation at all quinpirole concentrations used, RGS2 and RGSz1 showed only approximately 25-30% attenuation (FIG. 3B). In all three cases, inhibition by the RGS proteins persisted despite application of high concentrations of quinpirole. The rank order of potency of the RGS inhibiton correlated with their in vitro GAP activities toward Gαi.

To further study the interaction between the amount of Gα proteins and RGS protein selectivity in vivo, RGS2 and RGS4 were co-transfected with a Gαq/i chimera in CHO-D2R cells. The chimera was a fusion protein and possessed all the structural motifs of Gαq except the last 5 amino acids, which were replaced with the last 5 amino acids of Gαi1. The last 5 C-terminal amino acids of Gα proteins are responsible for binding Gα to its cognate receptors (See, Conklin et al., (1993) Nature 363: 274-276). Thus, while bound to the activated D2R, the chimera could generate Gαq-mediated signaling events and be modulated by Gαq-selective RGS proteins. Quinpirole stimulation of the Gαq/i over-expressed in CHO-D2R cells markedly activated the SRE-reporter gene with maximal activation of about 20 fold (FIG. 3B). The result was consistent with reports that activated Gαq by itself is a potent activator of c-fos SRE (See, Fromm, et al., (1997) Proc. Natl. Acad. Sci. 94: 10098-1-1-3; Mao et al., (1998) J. Biol. Chem. 273: 27118-27123). When RGS2, a potent GAP for Gαq in vitro (See, Heximer et al., (1997) Proc. Natl. Acad. Sci. 14389-14393), was co-expressed with Gαq/i, an approximately 70% reduction in SRE activation was observed. Co-expression of Gαq/i with RGS4, also a potent GAP for Gαq, but less potent than RGS2 (See, Berman et al., (1996) Cell 86: 445-452; Hepler et al., (1997) Proc. Natl. Acad. Sci. 94: 428-432), showed about 60% reduction in SRE activation. The differential potentiation by Gαq/i on the RGS2 and RGS4 activity was statistically significant and correlated with each protein's in vitro Gαq GAP activities. Thus, the quantity of Gα proteins may govern the strength and selectivity of RGS proteins in attenuating G protein signaling in vivo.

### Example 8

### Map Kinases Participate in D2R-Induced SRE Activation

Transient expression of Gβ₁γ₂ in NIH3T3 cells resulted in activation of an SRE-reporter gene (See, Fromm et al., (1997) Proc. Natl. Acad. Sci. 94: 10098-10103). The mechanism of action is putatively the TCF-linked route since it is well known that Gβγ activates the classical MAP kinases Erk1/2 via the Ras-Raf-MEK pathway (See, Lopez-Ilasaca, (1998) Biochem. Pharma. 56: 269-277). Phosphorylated Erk1/2 translocate to the nucleus, where Erk1 phosphorylates Elk1 (Id.), thereby leading to the TCF-linked transactivation of c-fos SRE (See, Shaw et al., (1989) Cell 56: 563-572; Treisman, (1994) Curr. Opin. Genet. Dev. 4: 96-101; Kortenjann et al., (1994) Mol. Cell. Biol. 14, 4815-4824). To address the contribution of Erk1/2 in the Gβγ-mediated SRE activation in CHO cells, CHO-D2R cells were treated with 25 nM of MEK inhibitor PD098059. Quinpirole stimulation resulted in phosphorylation of Erk1/2, which was completely suppressed by PD098059 treatment. However, cells treated with PD098059 showed only about 50% diminution of quinpirole-stimulated SRE activity as compared to cells treated with vehicle. Thus, in addition to the Ras-MAPK pathway, other signaling molecules may be involved in the Gβγ-mediated SRE activation in CHO cells.

### Example 9

### The Rho Family of Small G Proteins was Required for D2R-Induced SRE Activation

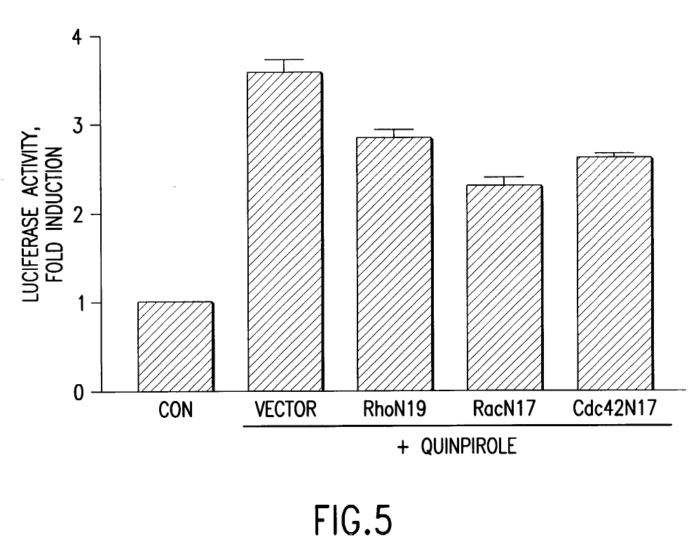

Small G proteins of the Rho family have been shown to activate the c-fos SRE (See, Hill et al., (1995) Cell 81: 1159-1170). A study was conducted to determine whether Gβγ signaling to the SRE in CHO cells was mediated in part via these small G proteins. CHO-D2R celis were transiently transfected with the dominant-negative mutants of RhoA, Rac1; and Cdc42, representatives of Rho family members. The mutants were generated through substitution of Thr19 of RhoA, Thr17 of Rac1, and Thr17 of Cdc42 with Asn. The analogous mutation in the related small GTPase Ras increased its affinity for GDP. The mutation resulted in sequestration of guanine nucleotide exchange factors (GEFs), making them unavailable for activation of endogenous Ras and thereby blocking downstream signaling events. RhoN19, RacN17, and CdcN17 have similarly been shown to function as dominant negative molecules (See, Coso et al., (1995) Cell 81: 1137-1146; Kozma et al., (1995) Mol. Cell. Biol. 15, 1942-1952; Minden et al., (1995) Cell 81: 1147-1157). Transfection of the respective dominant-negative mutants in CHO-D2R cells suppressed quinpirole-stimulated SRE activation (FIG. 5). Transfection of the C. botulinum C3 transferease, which inactivates Rho by ADP ribosylation of Asn 41 (See, Hill, (1994) Cell 81: 1159-1170), diminished the SRE activation as well. All three members of the Rho family were involved in the Gβγ signaling to the SRE in CHO cells.

### Example 10

### PI₃-K Was not Required for D2R-Initiated SRE Activation

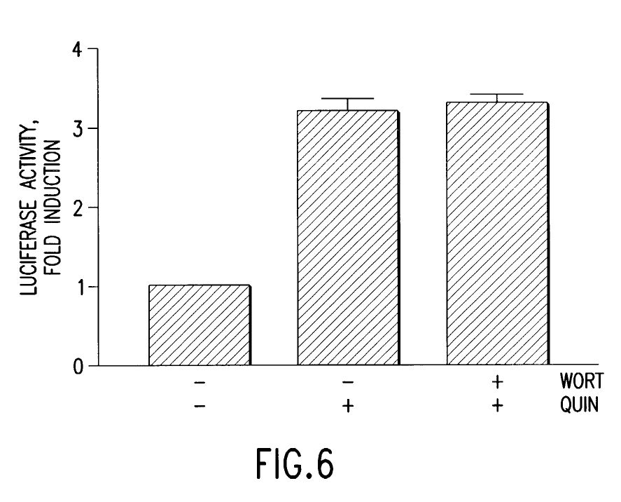

Gβγ activates PI₃-Kγ (See, Stephens et al., (1995) Cell 77: 83-93), and Rac has been shown to be downstream of PI₃-Kγ in Gβγ-mediated cytoskeletal reorganization (See, Ma et al., (1998) Mol. Cell. Biol. 18: 4744-4751). To address the involvement of the PI₃ kinase pathway in the Gβγ-mediated nuclear activation, CHO-D2R cells were treated with the PI₃-K inhibitor wortmannin (50 nM) prior to measurement of SRE activity. As shown in FIG. 6, quinpirole (100 nM) stimulation elicited phosphorylation of Akt, a downstream serine/threonine kinase, indicating that the quinpirole induced PI₃-K activation in CHO-D2R cells. Treatment of cells with wortmannin diminished Akt phosphorylation to its basal level. However, blockade of the PI₃-K activity did not alter the magnitude of the SRE activation, thereby ruling out a role for PI₃-K as a mediator of Gβγ signaling to the SRE. There is evidence to show that Gβγ can also activate the Ras-MAPK pathway via PI₃-Kγ (See, Lopez-Ilasaca et al., (1997) Science 275: 394-397). Western blot analysis of wortmannin-treated cells showed no inhibition of the Erk1/2 phosphorylation by wortmannin, suggesting that PI₃-K was not required for quinpirole-stimulated SRE activation in CHO cells.

### Example 11

### Implications and Discussion

Stimulation of Gq-coupled receptors or expression of activated Gαq and Gα_{12/13} induced SRE activation and cellular transformation (See, Fromm et al., (1997) Proc. Natl. Acad. Sci. 94: 10098-10103). The mechanism of action is linked to the small G protein Rho, because expression of C3 exoenzyme, a specific Rho inhibitor, abolished Gαq or Gα_{12/13}-induced SRE activation as well as transformation phenotypes (See, Fromm et al., (1997) Proc. Natl. Acad. Sci. 94: 10098-10103, Mao et al., (1998) J. Biol. Chem. 273: 27118-27123). CHO cells that stably express D2R provided evidence for a Gi-coupled receptor in mediating SRE activation (FIGS. 1 and 2). Moreover, quinpirole-stimulated SRE activation was completely abolished by expression of the Gβγ scanvanger βARKct, thus indicating a Gβγ-initiated event. This finding is consistent with the notion that expression of Gβγ induced SRE activity, while expression of constitutively active Gαi or Gαo failed to activate SRE (See, Fromm et al., (1997) Proc. Natl. Acad. Sci. 94: 10098-10103, Mao et al., (1998) J. Biol. Chem. 273: 27118-27123). Notably, Mao et al. were unable to observe the link between an agonist-induced D2R activation and the SRE-reporter activity in 293 cells.

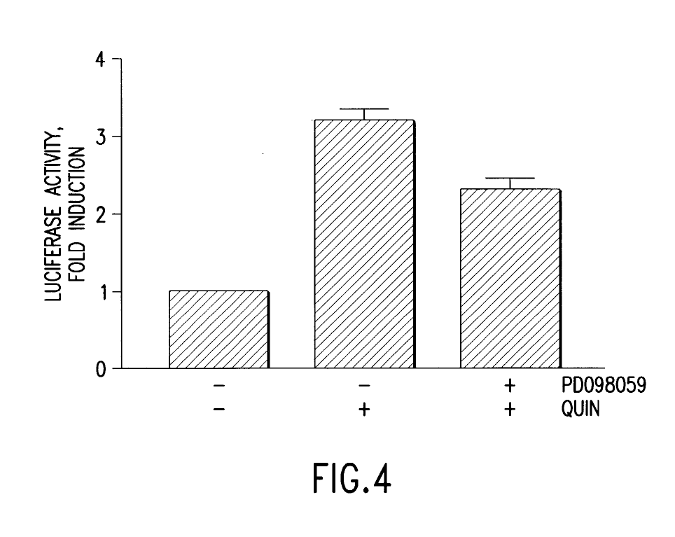

Gβγ-induced SRE activation likely involves the TCF-linked pathway because Gβγ is a well charaterized activator of the Ras-Raf-Erk pathway (See, Lopez-Ilasaca, (1998) Biochem. Pharma. 56: 269-277). Inhibition of Erk activation by PD098059 only partially suppressed quinpirole-stimulated SRE activation in CHO-D2R cells (FIG. 4), suggesting that, in addition to Erk1/2, other signaling molecules are involved. Expression of dominant negative mutants of the Rho family members diminished quinpirole-induced SRE activation as well (FIG. 5). Little is known about Gβγ activating the Rho family members. Gβγ may act through PI₃-Kγ to regulate Rac-dependent cytoskeletal reorganization (See, Ma et al., (1998) Mol. Cell. Biol. 18: 4744-4751). However, treating cells with wortmannin, which abolishes quinpirole-stimulated activation of the PI₃-K pathway, did not diminish SRE activation (FIG. 6). Thus, PI₃-K, though activated by quinpirole, did not appear to impact the Rho family-mediated transcriptional activity of SRE in CHO cells.

Members of the Rho family have been found to regulate the SRE-dependent gene transcription (See, Hill et al., (1995) Cell, 81: 1159-1170). Rho activates SRE via the transcriptional factor SRF-linked pathway, but the intermediary molecules linking Rho to SRF have not yet been identified. Rac and Cdc42 regulate gene transcription by activating the c-Jun N-terminal kinase (JNK) and p38 stress-induced kinase via a cascade of kinase-mediated phosphorylation events (See, Coso et al., (1995) Cell 81: 1137-1146; Minden et a., (1995) Cell 81: 1147-1157). Like family member Erk1, activated JNK and p38 translocate to the nucleus, where they phosphorylate transcription factor Elk1. Thus, Rac and Cdc42 could potentially mediate the quinpirole-stimulated SRE activation via the TCF-linked route. However, an endogenous level of either of the kinases in CHO cells was detected by Western blot. Thus, the significance of JNK and p38 in Gβγ to SRE signaling is uncertain. In Swiss 3T3 cells, there is a hierarchical order to the Rho family members in mediating cytoskeletal changes, with Cdc42 able to activate Rac, which, in turn, can activate Rho (See, Nobes et al., (1995) Cell 81: 53-62). Expression of dominant negative Rho or C3 exoenzyme blocks the Rac-induced the c-fos SRE activation in fibroblasts, thus placing Rac upstream of Rho in the signaling pathway (See, Kim et al., (1997) FEBS Lett. 415: 325-328).

Using the CHO-D2R cell system as a paradigm and the SRE activation as the signaling endpoint, RGS2, RG4, and RGSz attenuated quinpirole-stimulated SRE activation (FIG. 3). These RGS proteins are composed primarily of the RGS domain and do not contain additional protein-protein interaction motifs found in larger RGS proteins, which may link them to other signaling networks (See, Hepler (1999) Trends Pharma. Sci. 20: 376-382; De Vries et al., (1999) Trends Cell Biol. 9: 138-143). Thus, the attenuation is most likely due to the Gα GAP activity of the RGS proteins. Furthermore, over-expression of Gαi preferentially potentiated the inhibitory effect of RGS4 while over-expression of Gαq/i chimera potentiated the function of both RGS2 and RGS4. Because the Gα potentiation correlated with the selectivity of these RGS proteins, it is likely that attenuation of D2R-induced SRE activation is attributed to the Gα GAP activity of the RGS proteins.

All three RGS proteins in this study displayed an inhibition on the Gi-coupled SRE activation (FIG. 2). RGS2, a Gαq GAP in vitro, apparently inhibits Gi-coupled events (See, Ingi et al., (1998) J. Neurosci. 18: 7178-7188; Potenza et al., (1999) J. Pharma. Exp. Thera. 291: 482-491). Similarly, a blockade of a Gi-coupled MAPK kinase activation by RGSz1, a Gαz specific GAP (See, Wang et a., (1997) J. Biol. Chem. 273: 26014-26025 ; Click et al., (1997)) was observed. All three RGS proteins in this study, each with differential Gαi GAP activities, attenuated equally well quinpirole-stimulated SRE activation (FIG. 2), leaving open the question as to which factors govern the selectivity of RGS proteins in vivo. RGS selectivity may reside at several levels, such as differential tissue distribution (See, Gold et al., (1997) J. Neurosci. 17: 8024-8037), subcellular localization (See, Chatterjee et al., (2000) J. Biol. Chem. 275: 24013-24021), posttranslational modification (See, Ogier-Denis et al., (2000) J. Biol. Chem. 275: 39090-39095; Benzing et al., (2000) J. Biol. Chem. 275: 28167-28172 and receptor-G protein interaction (See, Xu et al., (1999) J. Biol. Chem. 274: 3549-3556). The three RGS proteins used in this study, when co-expressed with Gαi1, exhibited differential degrees of attenuation on quinpirole-stimulated SRE activation with RGS4, the strongest Gαi GAP, showing the strongest effect (FIG. 3). Thus, in addition to other factors that may contribute to the selectivity of RGS proteins, the quantity of G proteins in cells is a contributing factor.

In contrast to an increased Gαq-mediated transcriptional activation when wild-type Gαq is expressed in cells (See, Xie et al., (2000) J. Biol. Chem. 275: 24907-24914), a modest reduction of quinpirole-stimulated SRE activation was consistently observed when Gαi1 was transfected in cells (FIG. 3A). One explanation for this observation could be that while adding exogenous Gαi may increase both pools of the GTP-bound and GDP-bound Gαi in cells, the GTP-bound Gαi does not signal to the SRE (FIG. 1) (See, Fromm et al., (1997) Proc. Natl. Acad. Sci, 94: 10098-10103; Mao et al., (1998) J. Biol. Chem. 273: 27118-27123) while the GDP-bound Gαi terminates Gβγ signaling. Thus, providing exogenous Gαi to cells only results in negative regulation of Gβγ-initiated signaling events, hence reduction in the agonist-induced SRE activation was observed. Nevertheless, an even stronger attenuation of SRE activation by RGS proteins was observed when cells are co-transfected with Gαi. This observation could be explained by the GAP activity of RGS proteins, which shifts the equilibrium between the GTP-bound and GDP-bound Gαi further toward the GDP-bound form. Accordingly, the more potent a Gαi GAP is, the more pronounced inhibition by an RGS protein would be observed as shown in FIG. 3B.

Transfection of a Gαq/i chimera markedly potentiated quinpirole-stimulated SRE activation (FIG. 3C), which was expected because Gαq by itself activates SRE pathway. (See, Fromm et al., Proc. Natl. Acad. Sci. (1997) 94: 10098-10103; Mao et al., J. Biol. Chem. (1998) 273: 27118-27123). Gαq-induced SRE activation is mediated through the SRF-linked pathway (See, Fromm et al., Proc. Natl. Acad. Sci. (1997) 94: 10098-10103; Mao et al., J. Biol. Chem. (1998) 273: 27118-27123) and the Gβγ-induced SRE is mediated in part through the TCF-linked route (FIG. 4). Thus, the substantial induction of the SRE activity upon Gαq/i transfection likely resulted from the synergistic effect of the two transcriptional factors (SRF and TCF) on the c-fos SRE (See, Hill et al., (1995) Cell 81: 1159-1170). In fact, a much lower level of quinpirole-stimulated SRE activation was observed if cells were co-transfected with Gαq/i and βARKct, with the latter suppressing signaling input from the Gβγ-TCF pathway. Prolonged stimulation of Gαq-coupled receptors results in cellular transformation, a process dependent on the SRE activity (See, Fromm et al., Proc. Natl. Acad. Sci. (1997) 94: 10098-10103). RGS proteins, which attenuate signaling emanating from both Gαq and Gαγ, would be an efficient inhibitor in curbing prolonged GPCR activation under pathological conditions.

What is claimed is:

1. A method of assessing the efficacy of a test compound for inhibiting a GPCR-related disorder in a subject, the method comprising:
a) contacting a test cell with one of a plurality of test compounds in the presence of a GPCR agonist, wherein said test cell comprises:
i) a GPCR;
ii) a RGS protein;
iii) a corresponding Gα protein, expressed at a level capable of attenuating GPCR-signaling by at least 50% as compared to a cell without said Gα protein expression level; and
iv) a reporter gene;
b) detecting the expression of the reporter gene in the test cell contacted by a test compound; and
c) comparing the expression of the reporter gene in the test cell contacted by the test compound with the expression of the reporter gene in a test cell contacted by the agonist in the absence of the test compound,
wherein a substantially increased level of expression of the reporter gene in the test cell contacted by the test compound and the GPCR agonist, relative to the expression of the reporter gene in the test cell contacted by the GPCR agonist in the absence of the test compound, is an indication that the test compound is efficacious for inhibiting the GCPR-related disorder in the subject.

2. The method of claim 1; wherein the GPCR-related disorder is selected from the group consisting of neuropsychiatric disorders, cardiovascular disorders and inflammation.

3. The method of claim 1, wherein the GPCR is selected from the group consisting of D2 receptor, M2 receptor, 5HT1A receptor, Edg1 receptor and Bradykinin receptor.

4. The method of claim 1, wherein the RGS protein is selected from the group consisting of GAIP, RGSz1, RGS1, RGS2, RGS3, RGS4, RGS5, RGS6, RGS7, RGS8, RGS9, RGS10, RGS11, RGS13, RGS14, RGS16, RGS17, D-AKAP2, p115RhoGEF, PDZ-RhoGEF, bRET-RGS, Axin, and mCONDUCTIN.

5. The method of claim 1, wherein the reporter gene is selected from the group consisting of SRE-Luciferase, SRE-LacZ, SRE-CAT and CRE-Luciferase.

6. The method of claim 1, wherein the Gα protein is selected from the group consisting of Gαi and Gαq.

7. The method of claim 1, wherein the Gα protein is a chimeric protein.

8. A method of assessing the efficacy of a test compound for inhibiting a GPCR-related disorder in a subject, the method comprising the step of comparing:
a) expression of a RGS protein in the presence of Gα in a first cell sample, wherein the first cell sample is exposed to the test compound, and
b) expression of a RGS protein in the presence of Gα in a second cell sample, wherein the second cell sample is not exposed to the test compound,
wherein a substantially decreased level of expression of the RGS protein in the first sample, relative to the second sample, is an indication that the test compound is efficacious for inhibiting the GPCR-related disorder in the subject.

9. The method of claim 8, wherein the GPCR-related disorder is selected from the group consisting of neuropsychiatric disorders and cardiovascular disorders.

10. The method of claim 8, wherein the RGS protein is selected from the group consisting of GAIP, RGSz1, RGS1, RGS2, RGS3, RGS4, RGS5, RGS6, RGS7, RGS8, RGS9, RGS10, RGS11, RGS13, RGS14, RGS16, RGS17, D-AKAP2, p115RhoGEF, PDZ-RhoGEF, bRET-RGS, Axin, and mCONDUCTIN.

11. The method of claim 8, wherein the Gα protein is selected from the group consisting of Gαi and Gαq.

12. A method of high-throughput screening for test compounds capable of inhibiting a RGS protein, the method comprising:
a) contacting a test cell with one of a plurality of test compounds in the presence of a GPCR agonist, wherein the test cell comprises:
i) a GPCR,
ii) a RGS protein,
iii) a corresponding Gα protein expressed at a level capable of attenuating GPCR-signaling by at least 50% as compared to a cell without said Gα protein expression level, and
iv) a reporter gene;
b) detecting the expression of the reporter gene in the test cell contacted by a test compound relative to other test compounds; and
c) correlating the amount of expression level of the reporter gene with the ability of the test compound to inhibit RGS protein,
wherein increased expression of the reporter gene indicates that the test compound is capable of inhibiting the RGS protein.

13. The method of claim 12, wherein the GPCR is selected from the group, consisting of D2 receptor, M2 receptor, 5HTIA receptor, Edg1 receptor and Bradykinin receptor.

14. The method of claim 12, wherein the RGS protein is selected from the group consisting of GAIP, RGSz1, RGS1, RGS2, RGS3, RGS4, RGS5, RGS6, RGS7, RGS8, RGS9, RGS10, RGS11, RGS13, RGS14, RGS16, RGS17, D-AKAP2, p115RhoGEF, PDZ-RhoGEF, bRET-RGS, Axin, and mCONDUCTIN.

15. The method of claim 12, wherein the reporter gene is selected from the group consisting of SRE-Luciferase, SRE-LacZ, SRE-CAT and CRE-Luciferase.

16. The method of claim 12, wherein the Gα protein is selected from the group consisting of Gαi and Gαq.

17. The method of claim 12, wherein the Gα protein is a chimeric protein.

18. The method of claim 12, wherein the test compounds are bioactive agents or small molecules selected from the group consisting of naturally-occurring compounds, biomolecules, proteins, peptides, oligopeptides, polysaccharides, nucleotides and polynucleotides.

19. A method of high-throughput screening for test compounds capable of inhibiting a GPCR-related disorder in a subject, the method comprising:
a) combining a RGS protein, Gα, and a test compound;
b) detecting binding of the RGS protein and Gα in the presence of a test compound; and
c) correlating the amount of inhibition of binding between RGS and Gα with the ability of the test compound to inhibit the GPCR-related disorder,
wherein inhibition of binding of the RGS protein and Gα indicates that the test compound is capable of inhibiting the GPCR-related disorder.

20. The method of claim 19, wherein the test compounds are small molecules or bioactive agents, wherein the bioactive agents are selected from the group consisting of naturally-occurring compounds, biomolecules, proteins, peptides, oligopeptides, polysaccharides, nucleotides and polynucleotides.

21. The method of claim 19, wherein the Gα protein is selected from the group consisting of Gαi and Gαq.

22. A method of screening test compounds for inhibitors of a GPCR-related disorder in a subject, the method comprising the steps of:
a) obtaining a sample from a subject comprising cells;
b) contacting an aliquot of the sample with one of a plurality of test compounds;
c) detecting the expression level of a RGS protein and Gα in each of the aliquots; and
d) selecting one of the test compounds which substantially inhibits expression of the RGS protein in the aliquot containing that test compound, relative to other test compounds.

23. A method of screening test compounds for inhibitors of a GPCR-related disorder in a subject, the method comprising the steps of:
a) obtaining a sample from a subject comprising cells;
b) contacting an aliquot of the sample with one of a plurality of test compounds;
c) detecting the activity of a RGS protein and Gα in each of the aliquots; and
d) selecting one of the test compounds which substantially inhibits activity of a RGS protein in the aliquot containing that test compound, relative to other test compounds.

24. A method of screening for a test compound capable of interfering with the binding of a RGS protein and a Gα, the method comprising:
a) combining a RGS protein, a test compound, and a Gα;
b) determining the binding of the RGS protein and the Gα; and
c) correlating the ability of the test compound to interfere with binding,
wherein a decrease in binding of the RGS protein and the Gα in the presence of the test compound as compared to the absence of the test compound indicates that the test compound is capable of inhibiting binding.

25. A method of determining the severity of a GPCR-related disorder in a subject, the method comprising the step of comparing:
a) a level of expression of RGS protein in a sample from the subject; and
b) a normal level of expression of RGS protein in a control sample,
wherein an abnormal level of expression of RGS protein in the sample from the subject relative to the normal level of expression of RGS protein is an indication that the subject is suffering from a severe GPCR-related disorder.

26. The method of claim 25, wherein the control sample is collected from tissue substantially free of the GPCR-related disorder and the abnormal level of expression of RGS protein is by a factor of at least about 2 relative to the level of normal RGS expression.

27. A method of assessing the efficacy of a therapy for inhibiting a GPCR-related disorder in a subject, the method comprising the steps of comparing:
a) expression of a RGS protein in a first sample obtained from the subject prior to providing at least a portion of the therapy to the subject, and
b) expression of a RGS protein in a second sample following provision of the portion of the therapy,
wherein a substantially modulated level of expression of the RGS protein in the second sample, relative to the first sample, is an indication that the therapy is efficacious for inhibiting the GPCR-related disorder in the subject.

28. A method for diagnosing a GPCR-related disorder, the method comprising:
a) obtaining a sample from a subject comprising cells;
b) measuring the expression of RGS and Gα in the sample,
c) correlating the amount of RGS and Gα with the presence of a GPCR-related disorder,
wherein the substantially increased levels of RGS and Gα as compared to a control sample are indicative of the presence of GPCR-related disorder.

29. A method of treating a subject diagnosed with a GPCR-related disorder, the method comprising administering a composition comprising
a) a RGS inhibitor which specifically binds to a RGS protein,
b) a Gα inhibitor which specifically binds to a Gα protein; and
c) a pharmaceutically acceptable carrier.

30. A method of treating a subject diagnosed with a GPCR-related disorder, the method comprising administering a composition comprising:
a) an antisense oligonucleotide complementary to a RGS polynucleotide,
b) an antisense oligonucleotide complementary to a Gα polynucleotide; and
c) a pharmaceutically acceptable carrier.

31. A method of enhancing GPCR-signaling, the method comprising providing to cells of a subject an antisense oligonucleotide complementary to a RGS polynucleotide.

32. A method of inhibiting GPCR-signaling, the method comprising providing to cells of a subject an antisense oligonucieotide complementary to Gα.

33. A composition capable of inhibiting a GPCR-related disorder in a subject, the composition comprising a therapeutically effective amount of: a) a RGS inhibitor which specifically binds to a RGS protein and b) a Gα inhibitor which specifically binds to a Gα protein; and a pharmaceutically acceptable carrier.

34. A composition capable of inhibiting a GPCR-related disorder, the composition comprising a therapeutically effective amount of: a) an antisense oligonucleotide complementary to a RGS polynucleotide and b) an antisense oligonucleotide complementary to a Gα polynucleotide; and a pharmaceutically acceptable carrier.

35. A composition capable of inhibiting a GPCR-related disorder, the composition comprising a therapeutically effective amount of: a) a ribozyme which is capable of binding a RGS polynucleotide and b) a ribozyme which is capable of binding a Gα polynucleotide; and a pharmaceutically acceptable carrier.

36. A genetically engineered test cell comprising: i) a GPCR, ii) a RGS protein, iii) a corresponding Gα protein expressed at a level capable of attenuating GPCR-signaling by at least 50% as compared to a cell without said Gα protein expression level, and iv) a reporter gene, wherein at least one of the components (i)-(iv) is introduced into the cell.

37. A kit for determining the long term prognosis in a subject having a GPCR-related disorder, the kit comprising a first polynucleotide probe, wherein the probe specifically binds to a transcribed RGS polynucleotide, and a second polynucleotide probe, wherein the probe specifically binds to a transcribed Gα polynucleotide.

38. A kit for determining the long term prognosis in a subject having a GPCR-related disorder, the kit comprising a first antibody, wherein the first antibody specifically binds to a RGS polypeptide, and a second antibody, wherein the second antibody specifically binds to a corresponding Gα polypeptide.

39. A kit for assessing the suitability of each of a plurality of compounds for inhibiting a GPCR-related disorder in a subject, the kit comprising:
a) a plurality of test cells, wherein each test cell comprises:
i) a GPCR,
ii) a RGS protein,
iii) a corresponding Gα protein expressed at a level capable of attenuating GPCR-signaling by at least 50% as compared to a cell without said Gα protein expression level, and
iv) a reporter gene, and
b) an agonist for the GPCR.

40. A method of treating a subject diagnosed with a GPCR-related disorder, the method comprising administering a composition comprising:
a) a ribozyme which is capable of binding a RGS polynucleotide,
b) a ribozyme which is capable of binding a Gα polynucleotide; and
c) a pharmaceutically acceptable carrier.


SEQUENCE LISTING (3 sequences)

SEQ ID NO: 1 - DNA, 70 bp, Homo sapiens
gccaccatggaacagaagctgatctccgaagaggacctcaacggcatgcaaagtgctatgttcttggctg

SEQ ID NO: 2 - DNA, 66 bp, Homo sapiens
ccaccatggaacagaagctgatctccgaagaggacctcaacggcatgtgcaaagggcttgcaggtc

SEQ ID NO: 3 - DNA, 68 bp, Homo sapiens
ccaccatggaacagaagctgatctccgaagaggacctcaacggcatgggatcagagcggatggagatg